(19) United States
(12) Patent Application Publication
(10) Pub. No.: US 2024/0415839 A1
(43) Pub. Date: Dec. 19, 2024

(54) Identification and Treatment of Cancers Associated with RASGRF1 Gene Fusions

(71) Applicant: YALE UNIVERSITY, New Haven, CT (US)

(72) Inventor: Frederick Wilson, New Haven, CT (US)

(21) Appl. No.: 18/699,532

(22) PCT Filed: Oct. 7, 2022
(86) PCT No.: PCT/US2022/077786

§ 371 Date: Apr. 8, 2024

Related U.S. Application Data
(60) Provisional application No. 63/253,859, filed on Oct. 8, 2021.

(51) Int. Cl. A61K 31/519; A61P 35/00; C12Q 1/6869; C12Q 1/6886

(52) U.S. Cl. CPC A61K 31/519; A61P 35/00; C12Q 1/6869; C12Q 1/6886; C12Q 2600/106; C12Q 2600/156

(57) ABSTRACT

The disclosure provides compositions and methods for identifying a cancer associated with an RASGRF1 gene fusion in a subject. The disclosure also includes methods of treating a subject who has been pre-selected by detecting an RASGRF1 gene fusion in a sample obtained from the subject.

### CROSS-REFERENCE TO RELATED APPLICATIONS

The present application claims priority under 35 U.S.C. § 119(e) to U.S. Provisional Patent Application No. 63/253,859, filed Oct. 8, 2021, which is hereby incorporated by reference in its entirety herein.

### STATEMENT REGARDING FEDERALLY SPONSORED RESEARCH OR DEVELOPMENT

This invention was made with government support under CA204732 awarded by National Institutes of Health. The government has certain rights in the invention.

### BACKGROUND

The therapeutic landscape for patients with advanced non-small cell lung carcinoma (NSCLC) has been transformed over the past 15 years with the introduction of small molecule targeted therapies and immune-directed therapies. In recent years, the identification of actionable oncogenic drivers in NSCLC and development of an expanding repertoire of small molecule inhibitors of these drivers have provided new therapeutic opportunities for molecular subsets of NSCLC. For instance, there are now FDA-approved targeted therapies for patients with advanced NSCLC harboring activating alterations in EGFR, ALK, ROS1, BRAF, RET, MET, and NTRK. In addition, emerging selective inhibitors of KRAS G12C, ERBB2, and others are currently in clinical development. These activating driver alterations upregulate pathways promoting cell proliferation and survival including the MAP kinase and PI3K pathways.

Oncogenic drivers in NSCLC are generally mutually exclusive of one another, and many (including alterations involving EGFR, ALK, ROS1, RET, and NTRK) are more prevalent in NSCLC from individuals with little to no smoking history. The frequency of some of these oncogenic drivers in Asian, European, and Canadian never-smokers have been reported. However, some of the more recently recognized drivers (such as MET Exon 14 splice alterations or RET rearrangements) were not evaluated, and no known driver could be identified in up to 25% of never-smokers in these studies.

Therefore, there is a need in the art for the identification of oncogenic drivers associated with cancer and methods of identifying and treating patients with these drivers. The present disclosure satisfies this unmet need.

### BRIEF SUMMARY

As described herein, the present disclosure relates to compositions and methods for identifying a cancer associated with an RASGRF1 gene fusion in a subject in need thereof. The disclosure also relates to methods of treating a subject who has been pre-selected by detecting an RASGRF1 gene fusion in a sample obtained from the subject.

As such, in one aspect, the disclosure includes a method of treating a subject having cancer. In certain embodiments, the method comprises administering a treatment for cancer to a pre-selected subject, wherein the subject is pre-selected by determining that an RASGRF1 gene fusion is present in a sample obtained from the subject.

In another aspect, the disclosure includes a method of diagnosing cancer in a subject. In certain embodiments, the method comprises determining the presence or absence of an RASGRF1 gene fusion in a sample obtained from the subject, wherein the presence of an RASGRF1 gene fusion indicates that the subject has cancer.

In another aspect, the disclosure includes a method of identifying a subject having cancer who is responsive to a therapy directed against RASGRF1 or a pathway activated by RASGRF1. In certain embodiments, the method comprises determining the presence or absence of an RASGRF1 gene fusion in a sample obtained from the subject, wherein the presence of an RASGRF1 gene fusion indicates the subject is responsive to a therapy directed against RASGRF1 or a pathway activated by RASGRF1.

In certain embodiments, the sample is a tissue sample, blood sample, or a tumor sample.

In certain embodiments, the RASGRF1 gene fusion comprises at least a portion of the RASGRF1 gene fused to at least a portion of a transmembrane protein.

In certain embodiments, the transmembrane protein is selected from the group consisting of: OCLN, TMEM87A, SLC4A4, and TMEM154.

In certain embodiments, the transmembrane protein is OCLN. In certain embodiments, the transmembrane protein is TMEM87A. In certain embodiments, the transmembrane protein is SLC4A4. In certain embodiments, the transmembrane protein is TMEM154.

In certain embodiments, the RASGRF1 gene fusion comprises the nucleotide sequence set forth in SEQ ID NO: 11. In certain embodiments, the RASGRF1 gene fusion comprises the nucleotide sequence set forth in SEQ ID NO: 12. In certain embodiments, the RASGRF1 gene fusion comprises the nucleotide sequence set forth in SEQ ID NO: 13.

In certain embodiments, the cancer is non-small cell lung carcinoma, pancreatic cancer, and/or bone marrow cancer.

In certain embodiments of the above aspects, or any aspect or embodiment disclosed herein, the step of determining the presence or absence of the RASGRF1 gene fusion in the sample comprises detecting the presence or absence of RASGRF1 gene fusion in the sample by whole-exome sequencing, whole-transcriptome sequencing, RNA sequencing, fluorescence in situ hybridization, immunohistochemistry, and/or a combination thereof.

In certain embodiments of the above aspects, or any aspect or embodiment disclosed herein, the disclosure further comprises a step of selecting and/or administering a treatment to the subject identified as having cancer or the subject identified as responsive to a therapy directed against RASGRF1 or a pathway activated by RASGRF1.

In certain embodiments, the treatment or therapy comprises a MAP kinase inhibitor, a MEK inhibitor, a PI3K inhibitor, a tyrosine kinase inhibitor (TKI), an inhibitor of the Ras-GEF domain of RASGRF1, an inhibitor of the GEF family, an ERK inhibitor, or combinations thereof. In certain embodiments, the treatment or therapy comprises a MAP kinase inhibitor. In certain embodiments, the treatment or therapy comprises a MEK inhibitor. In certain embodiments, the treatment or therapy comprises a PI3K inhibitor. In certain embodiments, the treatment or therapy comprises a tyrosine kinase inhibitor (TKI). In certain embodiments, the treatment or therapy comprises a an inhibitor of the Ras-GEF domain of RASGRF1. In certain embodiments, the treatment or therapy comprises an inhibitor of the GEF family. In certain embodiments, the treatment or therapy comprises an ERK inhibitor.

In certain embodiments, the treatment or therapy is selected from the group consisting of: imatinib, gefitinib, erlotinib, dasatinib, sunitinib, adavosertib, lapatinib, efametinib, selumetinib, trametinib, cobimetinib, idelalisib, copanlisib, duvelisib, alpelisib, taselisib, perifosine, buparlisib, umbralisib, voxtalisib, pictilisib, BAY-293, BI 1701963, BI-3406, ulixertinib, LY3214996, CC-90003, AZD0364, S0859, and combinations thereof.

In certain embodiments, the treatment disrupts or prevents the RASGRF1 gene fusion.

In another aspect, the disclosure includes a kit for the diagnosis cancer or for the identification of a subject responsive to a therapy directed against RASGRF1 or a pathway activated by RASGRF1, the kit comprising at least one agent capable of specifically binding or hybridizing to a polypeptide or polynucleotide of an RASGRF1 fusion, and directions for using the agent for the diagnosis of cancer.

In certain embodiments of the above aspects, or any aspect or embodiment disclosed herein, the disclosure further comprising directions and/or materials necessary for detecting the presence of fusion between at least a portion of an RASGRF1 polypeptide or polynucleotide and at least a portion of a second polypeptide or polynucleotide.

In another embodiment, the fusion is between at least a portion of the RASGRF1 polypeptide or polynucleotide and at least a portion of an OCLN, a TMEM87A, a SLC4A4, or a TMEM154 polypeptide or polynucleotide.

### BRIEF DESCRIPTION OF THE DRAWINGS

The following detailed description of selected embodiments of the disclosure will be better understood when read in conjunction with the appended drawings. For the purpose of illustrating the disclosure, selected embodiments are shown in the drawings. It should be understood, however, that the disclosure is not limited to the precise arrangements and instrumentalities of the embodiments shown in the drawings.

FIGS. 1A-1B depict an overview of genomic characterization of NSCLC from light or never-smokers. FIG. 1A: Schematic summarizing the approach for genomic characterization of 113 NSCLC tumors from the Yale Lung Cancer Biorepository. FIG. 1B: Clinical characteristics of study participants are shown.

FIG. 2 depicts a table of non-limiting identified oncogenic drivers.

FIGS. 3A-3G depict non-limiting identification and characterization of RASGRF1 fusions. FIG. 3A: Schematic depicting a gene rearrangement identified from YLCB345 that generates an in-frame fusion joining Exon 5 of OCLN with Exon 15 of RASGRF1 to generate OCLN-RASGRF1. FIG. 3B: Number of reads mapping to the 3′ portion of RASGRF1 preserved in OCLN-RASGRF1 are shown for the 26 NSCLCs subjected to RNAseq. Data is displayed in fragments per kilobase of transcript per million mapped reads (FPKM). YLCB345 is shown in red. FIG. 3C: Schematic depicting a gene rearrangement identified from PaCaDD137 cells that generates an in-frame fusion joining Exon 23 of SLC4A4 with Exon 11 of RASGRF1 to generate SLC4A4-RASGRF1. FIG. 3D: RT-PCR amplification of the full-length OCLN-RASGRF1 fusion transcript from YLCB345 (345). A molecular weight marker is shown (M). FIG. 3E: As in FIG. 3D, except for SLC4A4-RASGRF1 amplified from PaCaDD137. FIG. 3F: Sanger sequencing of the segment of OCLN-RASGRF1 spanning the fusion breakpoint. FIG. 3G: As in FIG. 3F, except for SLC4A4-RASGRF1.

FIGS. 4A-4F depict that RASGRF1 fusions increase levels of GTP-RAS and promote cell transformation. FIG. 4A: Western immunoblotting of lysates from HEK 293T cells expressing GFP, SLC4A4-RASGRF1 (S-R), or OCLN-RASGRF1 (O-R) to assess cellular levels of GTP-RAS and MAPK activation (p-ERK). Molecular weight (left) is indicated in kDa. See Materials and Methods in Example 1 for description of GTP-RAS pulldown assay. FIG. 4B: Anchorage-independent growth of NIH3T3 cells expressing GFP, EML4-ALK, OCLN-RASGRF1, or SLC4A4-RASGRF1 in soft agar assays. Cells were grown in soft agar for 19 days. Images are representative of 3 independent experiments. FIG. 4C: Average colony area was quantified from images captured for 3 independent experiments and normalized to GFP. Standard error is shown. FIG. 4D: Western immunoblotting of lysates from NIH3T3 cells expressing GFP, SLC4A4-RASGRF1 (S-R), OCLN-RASGRF1 (O-R), and EML4-ALK (E-A). FIG. 4E: Proliferation of Ba/F3 cells expressing GFP or OCLN-RASGRF1 after withdrawal of IL-3 (Day 0) at the indicated timepoints is shown. FIG. 4F: Western immunoblotting of lysates from wild-type (WT) Ba/F3 cells cultured with 1 ng/mL IL-3 and Ba/F3 cells expressing OCLN-RASGRF1 (O-R) in the absence of IL-3.

FIGS. 5A-5B depict that Ba/F3 cells expressing OCLN-RASGRF1 and PaCaDD137 cells are sensitive to targeting of the MAP kinase pathway with the MEK inhibitor trametinib. FIG. 5A: Wild-type (WT) Ba/F3 cells or Ba/F3 cells expressing OCLN-RASGRF1 were exposed to trametinib at the indicated concentrations. After 4 days, cell viability was determined using Cell Titer-Glo. WT Ba/F3 cells were cultured in the presence of 1 ng/mL IL-3. FIG. 5B: PaCaDD137 cells were exposed to inhibitors at the indicated concentrations. After 6 days, cell viability was determined using Cell Titer-Glo.

FIGS. 6A-6B depict a non-limiting summary of RASGRF1 fusions. Functional domains of full-length RASGRF1 are shown at the top. Shown below are 4 reported RASGRF1 fusions. In each case, the 5′ fusion partner is a membrane-spanning protein, and the entire RAS-GEF catalytic domain of RASGRF1 is preserved in the fusion. The tumor type from which each RASGRF1 fusion was identified is indicated on the right. PH1, pleckstrin homology domain 1. CC, coiled coil domain. IQ, isoleucine-glutamine domain. DH, dbl-homology region. PH2, pleckstrin homology domain 2. REM, Ras-exchanger stabilization motif domain. NSCLC, non-small cell lung carcinoma. AML, acute myeloid leukemia.

FIGS. 7A-7B depict additional analyses of an overview of genomic characterization of lung adenocarcinomas (LUADs) from light or never-smokers. FIG. 7A. Established oncogenic drivers identified from 103 LUADs from light (<10 pack-year) or never-smokers in the YLCB. FIG. 7B. Frequency of oncogenic drivers identified in the 103 profiled LUADs in the YLCB is shown. Below, frequency of oncogenic drivers identified in 300 advanced LUADs from never-smokers in the MSK-IMPACT clinical sequencing cohort is shown. MSK-IMPACT data was accessed via the cBioPortal for Cancer Genomics (cbioportal dot org).

FIG. 8 depicts RT-PCR amplification of the full-length 3 kb OCLN-RASGRF1 (O-R) and 5.3 kb SLC4A4-RASGRF1 (S-R) fusion transcripts from YLCB Tumor 9 and PaCaDD137 cells, respectively. Control (ctrl) PCR with no template is shown for each primer pair. Bands corresponding to amplified O-R and S-R fusions are indicated by a single asterisk and double asterisk, respectively. A molecular weight marker (M) is shown.

FIGS. 9A-9B depict sequencing of RASGRF1 fusion breakpoints. FIG. 9A. Sanger sequencing of the segment of OCLN-RASGRF1 spanning the fusion breakpoint. FIG. 9B. As in 9A, except for SLC4A4-RASGRF1.

FIGS. 10A-10F depict RASGRF1 fusions increasing levels of GTP-RAS and promoting cell transformation. FIG. 10A. Western immunoblotting of lysates from HEK 293T cells expressing GFP, SLC4A4-RASGRF1 (S-R), OCLN-RASGRF1 (O-R), or IQGAP1-RASGRF1 (I-R) to assess cellular levels of GTP-RAS and ERK activation (p-ERK). Molecular weight (left) is indicated in kDa. FIG. 10B. Cell surface protein biotinylation and purification were performed using lysates from HEK 293T cells with ectopic O-R expression or PaCaDD137 cells expressing endogenous S-R. Western immunoblotting was performed with total lysate (T) and lysate after selection for biotinylated surface proteins (S). Molecular weight (left) is indicated in kDa. Bands corresponding to O-R and S-R are denoted by * and **, respectively. FIG. 10C. Tumor cell focus formation due to loss of contact inhibition of proliferating NIH3T3 cells expressing the indicated RASGRF1 fusions compared to GFP. Cells were grown for 21 days. Images are representative of foci observed in 3 independent experiments. White marker represents 100 mm. FIG. 10D. Western immunoblotting of lysates from NIH3T3 cells expressing GFP, S-R, O-R, or I-R.

FIG. 10E. Proliferation of Ba/F3 cells expressing GFP or OCLN-RASGRF1 after withdrawal of IL-3 (Day 0) is shown. FIG. 10F. Western immunoblotting of lysates from wild-type (WT) Ba/F3 cells cultured with 1 ng/mL IL-3 and Ba/F3 cells expressing O-R in the absence of IL-3.

FIGS. 11A-11H depict the observation that Ba/F3 cells expressing OCLN-RASGRF1 and PaCaDD137 cells are sensitive to targeting of the RAF-MEK-ERK pathway with the MEK inhibitor trametinib. FIG. 11A. Wild-type (WT) Ba/F3 cells or Ba/F3 cells expressing OCLN-RASGRF1 were exposed to trametinib at the indicated concentrations. After 4 days, cell viability was determined using Cell Titer-Glo. WT Ba/F3 cells were cultured in the presence of 1 ng/mL IL-3. Mean and standard error are shown for all cell viability experiments, and each experiment was performed 3 times. FIG. 11B. Western immunoblotting of lysates from Ba/F3 cells expressing OCLN-RASGRF1 treated with trametinib at the indicated concentrations for 24 hours following serum starvation for 4 hours. Cl PARP, cleaved PARP. FIG. 11C. As in A, except cells were exposed to pictilisib at the indicated concentrations. FIG. 11D. Western immunoblotting of lysates from PaCaDD137 cells transfected with non-targeting control siRNA (ctrl) or siRNAs targeting RASGRF1. FIG. 11E. Viability of PaCaDD137 cells assayed 7 days after transfection with the indicated siRNAs (normalized to control siRNA). Mean and standard error for 3 independent experiments are shown. *p<0.0001 by two-tailed t-test. FIG. 11F. PaCaDD137 cells were exposed to inhibitors at the indicated concentrations. After 5 days, cell viability was determined using Cell Titer-Glo. FIG. 11G. Western immunoblotting of lysates from PaCaDD137 cells treated with pictilisib or trametinib at the indicated concentrations for 48 hours. FIG. 11H. Xenografts of PaCaDD137 cells were generated in nude mice (n=20). Once tumors reached 100 mm³, trametinib (1 mg/kg) or vehicle was administered daily (n=10 each) via intraperitoneal injection. Mean and standard error are shown. *p<0.01; **p<0.0001 by two-tailed t-test.

FIGS. 12A-12D depict the observation that RNA-seq identifies oncogenic drivers in lung adenocarcinomas (LUADs) from the Yale Lung Cancer Biorepository. FIG. 12A. Oncogenic MET Exon 14 splice alterations are identified in the indicated LUADs using RNA-seq. The number of reads spanning the junction between Exons 13 and 15 are indicated for each tumor. FIG. 12B. Expression of ERBB2 is shown in fragments per kilobase of transcript per million mapped reads (FPKM) for the 25 tumors subjected to RNA-seq. Tumor 21 (red) showed 24× copy number gain of ERBB2 and 22.9-fold increased ERBB2 expression compared to the other sequenced tumors. FIG. 12C. Reads mapping to the 3′ portion of RET preserved in RET fusions were used to determine RET expression in FPKM for 25 LUADs. Tumors harboring RET fusions are indicated in red. FIG. 12D. As in C except for ALK expression. Tumors harboring ALK fusions are indicated in red.

FIGS. 13A-13B depict RASGRF1 expression from RNA-seq of cancer cell lines in the Cancer Cell Line Encyclopedia (CCLE). FIG. 13A. RASGRF1 expression in pancreatic ductal adenocarcinoma (PDAC) cell lines (n=52). Number of cell lines with the indicated expression level are shown. Values for expression reflect RNA-seq transcripts per million (TPM) gene expression using RSEM software, Log 2 transformed, and using a pseudo-count of 1. Data is from DepMap Public 20Q4 release. The boxed column corresponds to PaCaDD137 with RASGRF1 expression=2.06. FIG. 13B. As in FIG. 13A, except for all CCLE lines (n=1378) including PDAC.

FIG. 14 is the sequence of OCLN-RASGRF1 (SEQ ID NO: 11) cloned from Tumor 9 in the Yale Lung Cancer Biorepository. RASGRF1 sequence is underlined.

FIG. 15 is the sequence of SLC4A4-RASGRF1 (SEQ ID NO: 12) cloned from PaCaDD137 cells. RASGRF1 sequence is underlined.

FIGS. 16A-16C depict the assembly of IQGAP1-RASGRF1 cDNA. FIG. 16A. Sequence of forward PCR primer used to generate IQGAP1-RASGRF1. The primer includes the first two exons of IQGAP1 (shown in black) and a portion of Exon 15 of RASGRF1 (underline). As the fusion endpoint occurs at Exon 15 of RASGRF1 for both IQGAP1-RASGRF1 and OCLN-RASGRF1, the full-length OCLN-RASGRF1 cDNA was used as a template for PCR amplification using the same reverse primer used to amplify OCLN-RASGRF1 and SLC4A4-RASGRF1. A secondary PCR was then performed with a forward primer incorporating an attB recombination site for Gateway cloning (see Methods for details). FIG. 16B. PCR amplification of the 2.1 kb IQGAP1-RASGRF1 (I-R) fusion transcript. Control (ctrl) PCR with no template is shown. A molecular weight marker (M) is shown. FIG. 16C. Sanger sequencing of the segment of IQGAP1-RASGRF1 spanning the fusion breakpoint.

FIG. 17 is the sequence of IQGAP1-RASGRF1 cDNA. RASGRF1 sequence is underlined.

FIGS. 18A-18F depict the sensitivity of cells expressing RASGRF1 fusions or comparator cell lines to small molecule inhibitors. FIG. 18A. KRAS-mutant pancreatic ductal adenocarcinoma (PDAC) SU8686 cells (which lack an RASGRF1 fusion) were exposed to inhibitors at the indicated concentrations. After 6 days, cell viability was determined using Cell Titer-Glo. Mean and standard error are shown for all experiments, and each experiment was performed 3 times. FIG. 18B. Heat map demonstrating half maximal inhibitory concentration (IC50) values for 29 PDAC cell lines exposed to trametinib in the Genomics of Drug Sensitivity in Cancer Project (GDSC2 Release 8.3 dataset; cancerrxgene.org). Heat map was generated using Morpheus software. FIG. 18C. Wild-type (WT) Ba/F3 cells or Ba/F3 cells expressing OCLN-RASGRF1 (O-R) were exposed to trametinib at the indicated concentrations in the presence of 0, 100, or 500 nM pictilisib. After 4 days, cell viability was determined using Cell Titer-Glo. WT Ba/F3 cells were cultured in the presence of 1 ng/mL IL-3. FIG. 18D. PaCaDD137 cells were exposed to trametinib at the indicated concentrations in the presence of 0, 100, or 500 nM pictilisib. Cell viability was determined after 6 days. FIG. 18E. WT Ba/F3 cells or Ba/F3 cells expressing OCLN-RASGRF1 were exposed to BAY293 at the indicated concentrations. Cell viability was determined after 4 days. FIG. 18F. As in FIG. 18E, except with sunitinib.

FIGS. 19A-19C depict the observation that Trametinib impairs growth of PaCaDD137 xenografts in nude mice. FIG. 19A. Data from the experiment depicted in FIG. 4H is shown for each individual mouse in the experiment treated with drug vehicle (n=10). FIG. 19B. As in A, except for trametinib (n=10). FIG. 19C. At the end of the experiment, tumors were extracted from representative mice in the trametinib and vehicle experimental arms (n=3 each) to confirm inhibition of RAF-MEK-ERK signaling. Lysates were prepared and subjected to Western immunoblotting with the indicated antibodies.

FIG. 20 depicts a schematic depicting a gene rearrangement identified from a giant cell sarcoma in The Cancer Genome Atlas (ID #TCGA-FX-A3TO) that generates IQGAP1-RASGRF1 from an in-frame fusion joining Exon 2 of IQGAP1 with Exon 15 of RASGRF1.

### DETAILED DESCRIPTION

The present disclosure relates generally to diagnostic methods and markers, prognostic methods and markers, and therapy evaluators for cancers associated with an RASGRF fusion. In certain embodiments, the markers of the disclosure comprise an RASGRF1 gene wherein at least a portion of the RASGRF1 gene is fused to at least a portion of a second (“partner”) gene. In certain embodiments, the second gene encodes a transmembrane protein. In some embodiments, the second gene is occludin (OCLN), SLC4A4, TMEM154, and/or TMEM87A. In some embodiments, the RASGRF1 fusion results in increased expression of RASGRF1. Therefore, in some embodiments, the presence of an RASGRF1 fusion can be confirmed by measuring a sample obtained from a patient for an increase in RASGRF1 expression compared to a control.

Accordingly, in certain embodiments, the present disclosure relates to biomarkers of cancer associated with RASGRF1 fusion. In certain embodiments, the present disclosure relates to methods for diagnosis of cancers associated with RASGRF1 fusion. In certain embodiments, the present disclosure relates to methods of determining predisposition to cancers associated with RASGRF1 fusion. In certain embodiments, the present disclosure relates to methods of monitoring progression/regression of cancers associated with RASGRF1 fusion. In certain embodiments, the present disclosure relates to methods of assessing efficacy of compositions for treating cancers associated with RASGRF1 fusion. In certain embodiments, the present disclosure relates to methods of screening compositions for activity in modulating biomarkers of cancers associated with RASGRF1 fusion. In certain embodiments, the present disclosure relates to methods of treating cancers associated with RASGRF1 fusion. In certain embodiments, the present disclosure relates to other methods based on detection of biomarkers of cancers associated with associated with RASGRF1 fusion in a biological sample. In some embodiments, the sample is a sample of cells. In some embodiments, the sample is a tissue sample.

In certain embodiments, the markers of the disclosure are useful for screening/diagnosing lung cancer, pancreatic cancer, and bone marrow cancer. In some embodiments, the markers of the disclosure are useful for screening/diagnosing advanced non-small cell lung carcinoma (NSCLC), including NSCLC in non-smokers, never smokers, and/or patients with minimal smoking history (<10 packs/year).

The disclosure also provides a method for permitting refinement of disease diagnosis, disease risk prediction, and clinical management of patients who have a cancer associated with RASGRF1 fusion, risk factors for developing a cancer associated with RASGRF1 fusion, increased levels of RASGRF1 expression, or abnormalities in pathways associated with RASGRF1. That is, the biomarkers of the disclosure can be used as a marker for the disease state or disease risk. For example, the presence of the selective biomarkers of the disclosure permits refinement of disease diagnosis, disease risk prediction, and clinical management of patients being treated with agents that are associated with cancer.

The disclosure also provides a method of diagnosing, treating, and monitoring a cancer associated with RASGRF1 fusion.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, non-limiting methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of 20% or +10%, more preferably +5%, even more preferably +1%, and still more preferably +0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

“Amplification” refers to any means by which a polynucleotide sequence is copied and thus expanded into a larger number of polynucleotide sequences, e.g., by reverse transcription, polymerase chain reaction or ligase chain reaction, among others.

An “analyte”, as used herein refers to any substance or chemical constituent that is undergoing analysis. For example, an “analyte” can refer to any atom and/or molecule; including their complexes and fragment ions. The term may refer to a single component or a set of components. In the case of biological molecules/macromolecules, such analytes include but are not limited to: polypeptides, polynucleotides, proteins, peptides, antibodies, DNA, RNA, carbohydrates, steroids, and lipids, and any detectable moiety thereof, e.g. immunologically detectable fragments. In some instances, an analyte can be a biomarker.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some instances, hyperproliferative disorders are referred to as a type of cancer including but not limited to primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

The term “biomarker” or “marker” is a characteristic that is objectively measured and evaluated as an indicator of normal biological processes, pathological processes, or pharmacological responses to a therapeutic intervention. The biomarker can for example describe a substance whose detection indicates a particular disease state. The biomarker may be a peptide that causes disease or is associated with susceptibility to disease. In some instances, the biomarker may be a gene that causes disease or is associated with susceptibility to disease. In other instances, the biomarker is a metabolite. In any event, the biomarker can be differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker can be differentially present at a level that is statistically significant (i.e., a p-value less than 0.05 and/or a q-value of less than 0.10 as determined using either Welch's T-test or Wilcoxon's rank-sum Test).

By “capture reagent” is meant a reagent that specifically binds a nucleic acid molecule or polypeptide to select or isolate the nucleic acid molecule or polypeptide.

By “decreases” is meant a negative alteration of at least 10%, 25%, 50%, 75%, 100%, 200%, 300%, 400%, 500%, 1000%, or more.

By “detect” refers to identifying the presence, absence, level, or concentration of an agent.

By “detectable” is meant a moiety that when linked to a molecule of interest renders the latter detectable. Such detection may be via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. In certain embodiments, the animal is a mammal. In certain embodiments, the mammal is a human.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence driven by its promoter.

By “fragment” is meant a portion of a polypeptide or nucleic acid molecule. This portion contains, in certain embodiments, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increases” is meant a positive alteration of at least 10%, 25%, 50%, 75%, 100%, 200%, 300%, 400%, 500%, 1000%, or more.

As used herein, an “instructional material” includes a publication, a recording, a diagram, or any other medium of expression which can be used to communicate the usefulness of the compositions and methods of the disclosure. The instructional material of the kit of the disclosure may, for example, be affixed to a container which contains the nucleic acid, peptide, and/or composition of the disclosure or be shipped together with a container which contains the nucleic acid, peptide, and/or composition. Alternatively, the instructional material may be shipped separately from the container with the intention that the instructional material and the compound be used cooperatively by the recipient.

By “marker profile” is meant a characterization of the expression or expression level of two or more polypeptides or polynucleotides

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are in certain embodiments directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “quantitative trait” refer to a phenotype or characteristics of an individual that can be attributed to the effect two or more genes.

As used herein, “quantitative trait locus (QTL)” refers to a DNA sequence or segment located within the genome containing or linked to the genes that underlie a quantitative trait.

As used herein, “expression quantitative trait loci (eQTLs)” are genomic loci that regulate expression levels of mRNAs or proteins. The abundance of a gene transcript is directly modified by polymorphisms in regulatory elements that alter the level of a gene transcript. These can be mapped and the level of a gene transcript can be used as a quantitative trait. Mapping eQTLs is performed using standard QTL mapping methods that test the linkage between variation in expression and genetic polymorphisms. In certain embodiments, eQTL is determined by statistical regression of the genotype of an SNP and the expression for the transcript.

By “reference” is meant a standard or control condition. In certain embodiments, the level of gene expression in a tissue sample of a subject having cancer is compared to the gene expression in a tissue sample from a control subject.

“Sample” or “biological sample” as used herein means a biological material isolated from a subject, including any tissue, cell, fluid, or other material obtained or derived from the subject (e.g., a human). The biological sample may contain any biological material suitable for detecting the desired analytes, and may comprise cellular and/or non-cellular material obtained from the subject. In various embodiments, the biological sample may be obtained from the bone marrow, lungs, or pancreas. In particular embodiments, the biological sample is a tissue sample.

By “single nucleotide polymorphism” or “SNP” is meant a DNA sequence variation occurring when a single nucleotide in the genome differs between members of a biological species or paired chromosomes in an individual. SNPs are used as genetic markers for variant alleles.

By “specifically binds” is meant a compound or antibody that recognizes and binds a polypeptide of the disclosure, but which does not substantially recognize and bind other molecules in a sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In certain embodiments, such a sequence is at least 60%, 80%, 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e^{″3} and e^{″100} indicating a closely related sequence.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, in certain embodiments less than about 500 mM NaCl and 50 mM trisodium citrate, and in certain embodiments less than about 250 mM NaCl and 25 mM trisodium citrate. Low stringency hybridization can be obtained in the absence of organic solvent, e.g., formamide, while high stringency hybridization can be obtained in the presence of at least about 35% formamide, and in certain embodiments at least about 50% formamide. Stringent temperature conditions will ordinarily include temperatures of at least about 30° C., in certain embodiments of at least about 37° C., and in certain embodiments of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a non-limiting embodiment, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will in certain embodiments be less than about 30 mM NaCl and 3 mM trisodium citrate, and in certain embodiments less than about 15 mM NaCl and 1.5 mM trisodium citrate. Stringent temperature conditions for the wash steps will ordinarily include a temperature of at least about 25° C., in certain embodiments of at least about 42° C., and in certain embodiments of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS.

Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

A “therapeutic” treatment is a treatment administered to a subject who exhibits a sign or symptom of pathology, for the purpose of diminishing or eliminating that sign or symptom.

As used herein, “treating a disease or disorder” means reducing the frequency with which a sign or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) cancer, including alleviating signs and symptoms of cancer.

By “variant” as is meant a polynucleotide or polypeptide sequence that differs from a wild-type or reference sequence by one or more nucleotides or one or more amino acids.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### RASGRF Fusion Biomarkers

The present disclosure relates in part to the identification of an Ras protein-specific guanine nucleotide-releasing factor (RASGRF) fusion biomarker that is associated with cancer. In certain embodiments, the RASGRF fusion biomarker comprises an RASGRF1 gene fused to a second (partner) gene. In certain embodiments, at least a portion of the RASGRF1 GEF is fused to the second gene. In certain embodiments, the second gene encodes a transmembrane protein. In some embodiments, the second gene is occludin (OCLN), SLC4A4, TMEM154, and/or TMEM87A.

In certain embodiments, the fusion biomarker is OCLN-RASGRF1, comprising an in-frame fusion joining exons 1-5 of the OCLN gene with exons 15-28 of the RASGRF1 gene. In other embodiments, the fusion biomarker is SLC4A4-RASGRF1, comprising an in-frame fusion joining exons 1-23 of the SLC4A4 gene with exons 11-28 of the RASGRF1 gene. In other embodiments, the fusion biomarker is TMEM154-RASGRF1, comprising a fusion joining exons 1-6 of the TMEM154 gene with exons 15-28 of the RASGRF1 gene. In other embodiments, the fusion biomarker is TMEM87A-RASGRF1, comprising a fusion joining a portion of the TMEM87A gene to exons 8-28 of the RASGRF1 gene. While not wishing to be limited by theory, the RASGRF1 fusions disclosed herein feature a transmembrane protein as the 5′ fusion partner with the fusion occurring in a location predicted to anchor the RAS-GEF domain of RASGRF1 to the cell membrane within a carboxy-terminal cytoplasmic tail. As membrane association is required for RAS activation, the 5′ transmembrane fusion partner may serve a key role in precisely localizing the RAS-GEF catalytic domain to membrane-associated RAS for activation.

In some embodiments, the biomarker is found in biological sample obtained from a patient being screened for cancer. In certain embodiments, the biological sample is a sample of cells. In other embodiments, the biological sample is a tissue sample such as tissue from a lump found on the patient or tissue from a tumor/suspected tumor. In some embodiments, the tumor tissue does not harbor an established oncogenic driver. Exemplary established oncogenic drivers include, but are not limited to, activating alterations in EGFR, KRAS, ALK, ERBB2, RET, BRAF, or PIK3CA, copy number gain of ERBB2, RET rearrangements, and MET Exon 14 splice alterations.

In some embodiments, the disclosed biomarker provides an indication of cancer or an increased risk of developing cancer in a subject. The cancer can be any type of cancer known to a person of skill in the art. Exemplary types of cancers are described elsewhere herein. In some embodiments, the biomarker can be used for cancer screening and diagnosis. In certain embodiments, the biomarker is an OCLN-RASGRF1 or a TMEM87A-RASGRF1 gene fusion and can be used to screen and/or diagnose lung cancer. In certain embodiments, the lung cancer is non-small cell lung carcinoma (NSCLC). In certain embodiments, the biomarker is used to screen and/or diagnose NSCLC in a non-smoker, a never-smoker, or a light smoker (<10 packs/year). In other embodiments, the biomarker is a SLC4A4-RASGRF1 gene protein and can be used to screen and/or diagnose pancreatic cancer. In other embodiments, the biomarker is a TMEM154-RASGRF1 gene protein and can be used to screen and/or diagnose bone marrow cancer.

In some embodiments, the invention provides an isolated RASGRF1 gene fusion comprising the nucleotide sequence set forth in SEQ ID NO: 11. In some embodiments, the invention provides an isolated RASGRF1 gene fusion comprising the nucleotide sequence set forth in SEQ ID NO: 12. In some embodiments, the invention provides an isolated RASGRF1 gene fusion comprising the nucleotide sequence set forth in SEQ ID NO: 13.

### Methods of Screening for Cancer

In certain embodiments, the present disclosure includes a method of screening for cancer in a patient in need thereof, the method comprising the steps of: obtaining a sample from the patient and analyzing the sample for the presence or absence of an RASGRF1 fusion. In certain embodiments, if an RASGRF1 fusion is present, the subject is identified as having cancer or likely to develop cancer. In other embodiments, if an RASGRF1 fusion is present, the subject is identified as responsive to a therapy directed against RASGRF1 or a pathway activated by RASGRF1.

The step of obtaining a sample from the patient can use any method known to a person of skill in the art. In some embodiments, the sample is a tissue sample is taken from a lump or suspected tumor on the patient. In some embodiments, the sample is a sample of cells taken from the patient, such as cells obtained during a biopsy. In certain embodiments, the sample is obtained from the lung, pancreas, or bone marrow of the patient.

The step of analyzing the sample can use any method known to a person of skill in the art. In some embodiments, genomic DNA and/or RNA is isolated from the sample and analyzed using whole-exome sequencing and/or RNA sequencing. In some embodiments, the genomic DNA and/or RNA is analyzed for RASGRF1 gene fusions. In certain embodiments, the RASGRF1 gene fusion is OCLN-RASGRF1, TMEM87A-RASGRF1, SLC4A4-RASGRF1, TMEM154-RASGRF1, or a combination thereof.

The disclosed method can be used to screen for any type of cancer. Exemplary types of cancer are described elsewhere herein. In some embodiments, the cancer is a cancer wherein tumors exhibit RASGRF1 fusion. In certain embodiments, the method is used to screen for NSCLC, pancreatic cancer, or bone marrow cancer.

### Methods of Treating a Pre-Selected Patient

Although not wishing to be limited by theory, it is believed that the RASGRF1 fusion can lead to increased RASGRF1 expression, increased expression of the C-terminal RAS-GEF domain, increased levels of GTP-bound (active) RAS, activation MAP kinase signaling, activation of the PI3K pathway, promotion of cell transformation, or a combination thereof. Therefore, although not wishing to be limited by theory, RASGRF1 and/or pathways activated by RASGRF1, such as the MAP kinase and PI3K pathways, may be potential therapeutic targets in tumors comprising an RASGRF1 fusion. In other embodiments, the gene that fuses with RASGRF1 is a therapeutic target in tumors comprising an RASGRF1 fusion. In some embodiments, therapies targeting OCLN, TMEM87A, SLC4A4, and/or TMEM154 can be used to treat or prevent cancer associated with an RASGRF1 fusion.

In certain embodiments, the present disclosure includes a method of treating a pre-selected patient, the method comprising the step of administering to the patient a treatment or therapy. In certain embodiments, the therapy is directed against RASGRF1 or pathways activated by RASGRF1. In other embodiments, the treatment prevents and/or disrupts RASGRF1 fusion. In some embodiments, the treatment or therapy is selected from a MAP kinase inhibitor, a MEK inhibitor, a PI3K inhibitor, a tyrosine kinase inhibitor (TKI), an inhibitor of the Ras-GEF domain of RASGRF1, an inhibitor of the GEF family (such as an inhibitor SOS1), an ERK inhibitor, or combinations thereof. In some embodiments, therapies targeting the RTK pathway can be used to treat or prevent cancer associated with an RASGRF1 fusion. Exemplary RTK inhibitors include, but are not limited to, imatinib, gefitinib, erlotinib, dasatinib, sunitinib, adavosertib, lapatinib, and combinations thereof. In some embodiments, tumors associated with an RASGRF1 fusion disclosed herein can be treated with any MEK inhibitor known to a person of skill in the art. Exemplary MEK inhibitors include, but are not limited to, efametinib, selumetinib, trametinib, cobimetinib, and combinations thereof. In some embodiments, tumors associated with a fusion protein disclosed herein can be treated with any PI3K inhibitor known to a person of skill in the art. Exemplary PI3K inhibitors include, but are not limited to, idelalisib, copanlisib, duvelisib, alpelisib, taselisib, perifosine, buparlisib, umbralisib, voxtalisib, pictilisib, and combinations thereof. In some embodiments, tumors associated with a fusion protein disclosed herein can be treated with any inhibitors of the GEF family known to a person of skill in the art. In certain embodiments, the inhibitor of the GEF family is an inhibitor of SOS1. Exemplary SOS1 inhibitors include, but are not limited to, BAY-293, BI 1701963, BI-3406, and combinations thereof. In some embodiments, tumors associated with a fusion protein disclosed herein can be treated with any ERK inhibitor known to a person of skill in the art. In some embodiments, the ERK inhibitor targets ERK1 and/or ERK1 components of the MAP kinase pathway downstream of MEK. Exemplary ERK inhibitors include, but are not limited to, ulixertinib, LY3214996, CC-90003, FR 180204, XMD 8-92, TMCB, Pluripotin, TCS ERK 11e, ERK5-IN-1, DEL 22379, AX 15836, AZD0364, and combinations thereof.

In certain embodiments, the treatment to prevent and/or disrupt RASGRF1 fusion targets the partner of the RASGRF1 fusion. In some embodiments, the treatment targets a partner of RASGRF1 fusion selected from OCLN, TMEM87A, SLC4A4, and TMEM154. In one embodiment, the treatment comprises S0859 (PMID: 18204485), wherein this compound inhibits SLC4A4. In certain embodiments, the treatment disrupts the fusion between RASGRF1 and its partner. In other embodiments, the treatment prevents the fusion between RASGRF1 and its partner. In some embodiments, the patient is administered a treatment selected from trametinib, pictilisib, BAY-293, sunitinib, or a combination thereof.

In some embodiments, the patient is pre-selected by detecting an RASGRF1 gene fusion in a sample obtained from the patient. In some embodiments, the patient has already been screened for established oncogenic drivers. Exemplary established oncogenic drivers are described elsewhere herein.

In certain embodiments, the biomarker of the disclosure is an RASGRF1 fusion. In some embodiments, the RASGRF1 fusion is OCLN-RASGRF1, TMEM87A-RASGRF1, SLC4A4-RASGRF1, or TMEM154-RASGRF1. In certain embodiments, the biomarker of the disclosure is detected to have increased expression in a subject with cancer compared to a control sample. In certain embodiments, biomarker of the disclosure is detected to have increased expression in a subject with NSCLC, pancreatic cancer, or bone marrow cancer compared to a control sample.

In some embodiments, the method further comprises the step of administering to the patient a therapeutic to treat cancer, and/or to treat the symptoms associated with cancer. The therapeutic can be any therapeutic known to a person of skill in the art to treat cancer and/or symptoms associated with cancer. Exemplary therapeutics to treat cancer and/or treat the symptoms associated with cancer include but are not limited to, Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alemtuzumab, Alimta (Pemetrexed Disodium), Aliqopa (Copanlisib Hydrochloride), Alkeran (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPOX, Carac (Fluorouracil—Topical), Carboplatin, CARBOPLATIN-TAXOL, Carfilzomib, Carmustine, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin HydrochlorideDoxorubicin, Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride). Exemestane, 5-FU (Fluorouracil Injection), 5-FU (Fluorouracil—Topical), Fam-Trastuzumab Deruxtecan-nxki, Fareston (Toremifene), Farydak (Panobinostat), Faslodex (Fulvestrant), FEC, Fedratinib Hydrochloride, Femara (Letrozole), Filgrastim, Firmagon (Degarelix), Fludarabine Phosphate, Fluoroplex (Fluorouracil—Topical), Fluorouracil, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane 1131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, Ixempra (Ixabepilone), Jakafi (Ruxolitinib Phosphate), JEB, Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lomustine, Lonsurf (Trifluridine and Tipiracil Hydrochloride), Lorbrena (Lorlatinib), Lorlatinib, Lumoxiti (Moxetumomab Pasudotox-tdfk), Lupron (Leuprolide Acetate), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Mercaptopurine, Mesna, Mesnex (Mesna), Methotrexate, Methylnaltrexone Bromide, Midostaurin, Mitomycin, Mitoxantrone Hydrochloride, Mogamulizumab-kpkc, Monjuvi (Tafasitamab-cxix), Moxetumomab Pasudotox-tdfk, Mozobil (Plerixafor), Mustargen (Mechlorethamine Hydrochloride), Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perjeta (Pertuzumab), Pertuzumab, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoetin Alfa, Retevmo (Selpercatinib), Revlimid (Lenalidomide), Rheumatrex (Methotrexate), Ribociclib, R-ICE, Ripretinib, Rituxan (Rituximab), Rituxan Hycela (Rituximab and Hyaluronidase Human), Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomide, Temsirolimus, Thalidomide, Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trexall (Methotrexate), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), and combinations thereof.

In certain embodiments, a therapeutically effective amount of a composition comprising a treatment to prevent and/or disrupt RASGRF1 fusion is administered to the subject that is sufficient to treat the cancer. The composition may be administered systemically, for example, formulated in a pharmaceutically-acceptable buffer such as physiological saline. The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy. In particular embodiments, the treatment is administered orally, intravenously, or topically.

The amount of the therapeutic agent to be administered varies depending upon the manner of administration, the age and body weight of the patient, and with the clinical symptoms of the cancer. Generally, amounts will be in the range of those used for other agents used in the treatment of cancers, although in certain instances lower amounts will be needed because of the increased specificity of the agent. A composition is administered at a dosage that decreases effects or symptoms of a cancer as determined by a method known to one skilled in the art. In some embodiments wherein the treatment or therapy disclosed herein is FDA-approved for the same and/or other indications, the dosage administered to the patient is determined from the recommended dosage on the manufacturer's drug label. In other embodiments, the dosage administered to the patient is determined by early-phase clinical studies to identify maximum tolerated dose with activity against the target.

### Diagnostic Methods

The methods of the disclosure also include the use of a biomarker to detect cancer in a subject (e.g., a human subject). As described herein, RASGRF gene fusions to a second (partner) gene indicated the presence of cancer in a subject. In one aspect, the present disclosure relates to a method of determining the presence or absence of an RASGRF1 gene fusion in a sample obtained from the subject, wherein the presence of an RASGRF1 gene fusion indicates that the subject has cancer. In another aspect, the present disclosure relates to a method of identifying a subject having cancer who is responsive to a therapy directed against RASGRF1 or a pathway activated by RASGRF1, the method comprising determining the presence or absence of an RASGRF1 gene fusion in a sample obtained from the subject, wherein the presence of an RASGRF1 gene fusion indicates the subject is responsive to a therapy directed against RASGRF1 or a pathway activated by RASGRF1.

In some embodiments, the sample obtained from the subject is a biological sample. Biological samples include tissue samples (e.g., cell samples, biopsy samples), such as tissue from the lungs or pancreas or cell samples from bone marrow. Biological samples also include bodily fluids, including, but not limited to, blood, blood serum, plasma, saliva, and urine. Presence of a statistically significant level of a biomarker alone or in combination with one or more additional markers relative to a reference are considered a positive indicator of a cancer. In certain embodiments, the biological sample is a tissue sample. In certain embodiments, the biological sample is a blood sample. In some embodiments, the tissue sample is from a tumor or suspected tumor.

Any suitable method can be used to detect one or more of the markers described herein. Successful practice of the disclosure can be achieved with one or a combination of methods that can detect and, in certain embodiments, quantify the markers. These methods include, without limitation, hybridization-based methods, including those employed in biochip arrays, mass spectrometry (e.g., laser desorption/ionization mass spectrometry), fluorescence (e.g. sandwich immunoassay), surface plasmon resonance, ellipsometry and atomic force microscopy. Expression levels of markers are compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)”, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

In some embodiments, the presences or absence of an RASGRF gene fusion is determined by any next-generation sequencing (NGS) based approach known to a person of skill in the art. In some embodiments, the NGS based approach comprises whole-transcriptome RNA sequencing, whole-exome sequencing, or RNA sequencing of the biological sample obtained from the subject. In some embodiments, DNA and/or RNA-based methods of detecting RASGRF fusions could be developed and incorporated into existing NGS cancer gene panels, permitting the detection of an RASGRF gene fusion using a commercially available platform including, but not limited to, platforms developed for testing tissue from a tumor or suspected tumor (such as Foundation Medicine) and platforms developed for testing circulating tumor DNA from peripheral blood (such as Guardant). In another embodiment, the presence or absence of an RASGRF gene fusion is determined by fluorescence in situ hybridization (FISH), a DNA-based hybridization technique applied to cells from the biological sample to detect and identify gene rearrangements. In some embodiments, the biological sample analyzed by FISH is obtained from a tumor or a suspected tumor. In another embodiment, the presence or absence of an RASGRF gene fusion is determined by immunohistochemistry. In some embodiments, the immunohistochemistry uses antibodies that recognize the RASGRF protein (or the partner protein) to detect the presence of an RASGRFfusion and degree of RASGRF1 expression in tissue obtained from a tumor or suspected tumor. In some embodiments, the RASGRF gene fusion comprises an RASGRF1 gene.

Detection methods may include use of a biochip array. Biochip arrays useful in the disclosure include protein and polynucleotide arrays. One or more markers are captured on the biochip array and subjected to analysis to detect the level of the markers in a sample.

Markers may be captured with capture reagents immobilized to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In certain embodiments, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and nonimaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy.

Mass spectrometry (MS) is a well-known tool for analyzing chemical compounds. Thus, in certain embodiments, the methods of the present disclosure comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

In an additional embodiment of the methods of the present disclosure, multiple markers are measured. The use of multiple markers increases the predictive value of the test and provides greater utility in diagnosis, patient stratification and patient monitoring. The process called “Pattern recognition” detects the patterns formed by multiple markers greatly improves the sensitivity and specificity of clinical markers for predictive medicine. Subtle variations in data from clinical samples indicate that certain patterns of protein expression can predict phenotypes such as the presence or absence of a certain disease, a particular stage of disease-progression, or a positive or adverse response to drug treatments. In the present disclosure, additional markers may include those associated with established oncogenic drivers.

Expression levels and/or alterations in sequences of particular nucleic acids or polypeptides are correlated with a cancer associated with RASGRF fusion and thus are useful in diagnosis. Methods for measuring the presence or levels of polypeptide include immunoassay, ELISA, western blotting and radioimmunoassay. Oligonucleotides or longer fragments derived from a nucleic acid sequence described herein, antibodies that bind a polypeptide described herein, or any other method known in the art may be used to monitor presence or expression of a polynucleotide or polypeptide of interest. In certain embodiments, the presence of a marker of the disclosure in a sample obtained from a subject is indicative of the subject having a cancer, e.g., a associated with an RASGRF1 fusion, and/or the subject being responsive to a therapy directed against RASGRF1 or pathways activated by RASGRF1. The diagnostic methods described herein can be used individually or in combination with any other diagnostic method described herein for a more accurate diagnosis of the presence of cancer.

As indicated above, the disclosure provides methods for aiding diagnosis of cancer using one or more markers, as specified herein. These markers can be used alone, in combination with other markers in any set, or with entirely different markers in aiding diagnosis. The measurement of markers may also involve quantifying the markers to correlate the detection of markers with a diagnosis of cancer. Thus, if the amount of the markers detected in a subject being tested is different compared to a control amount (i.e., higher than the control), then the subject being tested has a higher probability of having cancer. The correlation may take into account the amount of the marker or markers in the sample compared to a control amount of the marker or markers (e.g., in normal subjects). A control can be, e.g., the average or median amount of marker present in comparable samples of normal subjects in normal subjects or in subjects such as where the disease or disorder is undetectable. The control amount is measured under the same or substantially similar experimental conditions as in measuring the test amount. As a result, the control can be employed as a reference standard, where the normal (non-disease) phenotype is known, and each result can be compared to that standard, rather than re-running a control.

Accordingly, a marker profile may be obtained from a subject sample and compared to a reference marker profile obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate diagnosis of cancer.

In certain embodiments of the methods of qualifying a disorder, the methods further comprise managing subject treatment based on the status. The disclosure also provides for such methods where the markers (or specific combination of markers) are measured again after subject management. In these cases, the methods are used to monitor the status of the disorder or progression of the disorder.

Any marker, individually, is useful in aiding in the diagnosis of cancer. First, the selected marker is detected in a subject sample using the methods described herein. Then, the result is compared with a control that distinguishes disorder status from non-disorder status. As is well understood in the art, the techniques can be adjusted to increase sensitivity or specificity of the diagnostic assay depending on the preference of the diagnostician.

In embodiments wherein the presence of an RASGRF1 gene fusion has been determined, the subject is identified as having a cancer which is responsive to a therapy directed against RASGRF1 or a pathway activated by RASGRF1. The diagnosis of cancer associated with an RASGRF gene fusion can be used to inform treatment selection. Treatments for such cancers include, without limitation, treatment that prevent/disrupt RASGRF fusion such as a MAP kinase inhibitor, a MEK inhibitor, a PI3K inhibitor, a tyrosine kinase inhibitor (TKI), an inhibitor of the Ras-GEF domain of RASGRF, an inhibitor of the GEF family (such as an inhibitor SOS1), an ERK inhibitor, or combinations thereof. In certain embodiments, the treatment to prevent and/or disrupt RASGRF fusion targets the partner of the RASGRF fusion. Exemplary treatments to prevent and/or disrupt RASGRF fusion are disclosed elsewhere herein. Additionally, treatment for cancers involving an RASGRF fusion may include targeted therapies that decrease or eliminate the expression of any of the nucleic acid molecules or polypeptides of the genes responsible for the unique transcriptomes described herein. The treatment may additionally include therapeutics to treat cancer and/or symptoms associated with cancer. Exemplary therapeutics to treat cancer and/or symptoms associated with cancer are disclosed elsewhere herein.

While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, non-limiting methods of the present disclosure comprise the measurement of more than one marker.

### Microarrays

As reported herein, a number of biomarkers have been identified that are associated with cancer. In particular, the disclosure provides diagnostic methods and compositions useful for identifying an expression profile that identifies a subject as a cancer associated with an RASGRF fusion. Such assays can be used to measure an alteration in the level and/or sequence of a gene transcript or polypeptide encoded by the transcript.

The polypeptides and polynucleotides of the disclosure are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14: 1675-1680, 1996), and Schena, et al. (Proc. Natl. Acad. Sci. 93: 10614-10619, 1996), herein incorporated by reference. Methods for making polypeptide microarrays are described, for example, by Ge (Nucleic Acids Res. 28: e3 . . . i-e3. vii, 2000), MacBeath et al., (Science 289: 1760-1763, 2000), Zhu et al. (Nature Genet. 26:283-289), and in U.S. Pat. No. 6,436,665, hereby incorporated by reference.

### Nucleic Acid Microarrays

To produce a nucleic acid microarray, oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, in certain embodiments as a bodily fluid (such as blood, blood serum, plasma, saliva, urine, seminal fluids, and ejaculate) or tissue sample (e.g. a tissue sample obtained by biopsy). For some applications, cultured cells or other tissue preparations may be used. The mRNA is isolated according to standard methods, and cDNA is produced and used as a template to make complementary RNA suitable for hybridization. Such methods are known in the art. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, in certain embodiments less than about 500 mM NaCl and 50 mM trisodium citrate, and in certain embodiments less than about 250 mM NaCl and 25 mM trisodium citrate. Low stringency hybridization can be obtained in the absence of organic solvent, e.g., formamide, while high stringency hybridization can be obtained in the presence of at least about 35% formamide, and in certain embodiments at least about 50% formamide. Stringent temperature conditions will ordinarily include temperatures of at least about 30° C., in certain embodiments of at least about 37 C, and in certain embodiments of at least about 42° C.

Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/μl denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will in certain embodiments be less than about 30 mM NaCl and 3 mM trisodium citrate, and in certain embodiments less than about 15 mM NaCl and 1.5 mM trisodium citrate. Stringent temperature conditions for the wash steps will ordinarily include a temperature of at least about 25° C., in certain embodiments of at least about 42° C., and in certain embodiments of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct nucleic acid sequences simultaneously (e.g., Heller et al., Proc. Natl. Acad. Sci. 94:2150-2155, 1997). In certain embodiments, a scanner is used to determine the levels and patterns of fluorescence.

### Polymerase Chain Reaction (PCR)

Polymerase chain reaction (PCR) is widely known in the art. For example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2nd Ed., Springer-Verlag (New York: 1997), the disclosures of which are incorporated herein by reference, describe processes to amplify a nucleic acid sample target using PCR amplification extension primers which hybridize with the sample target. As the PCR amplification primers are extended, using a DNA polymerase (in certain embodiments thermostable), more sample target is made so that more primers can be used to repeat the process, thus amplifying the sample target sequence. Typically, the reaction conditions are cycled between those conducive to hybridization and nucleic acid polymerization, and those that result in the denaturation of duplex molecules.

In the first step of the reaction, the nucleic acid molecules of the sample are transiently heated, and then cooled, in order to denature double stranded molecules. Forward and reverse primers are present in the amplification reaction mixture at an excess concentration relative to the sample target. When the sample is incubated under conditions conducive to hybridization and polymerization, the primers hybridize to the complementary strand of the nucleic acid molecule at a position 3′ to the sequence of the region desired to be amplified that is the complement of the sequence whose amplification is desired. Upon hybridization, the 3′ ends of the primers are extended by the polymerase. The extension of the primer results in the synthesis of a DNA molecule having the exact sequence of the complement of the desired nucleic acid sample target. The PCR reaction is capable of exponentially amplifying the desired nucleic acid sequences, with a near doubling of the number of molecules having the desired sequence in each cycle. Thus, by permitting cycles of hybridization, polymerization, and denaturation, an exponential increase in the concentration of the desired nucleic acid molecule can be achieved.

The methods of the present disclosure involve amplifying regions of a polynucleotide with high fidelity using a thermostable DNA polymerase having 3′→5′ exonuclease activity. As defined herein, “3′→5′ exonuclease activity” refers to the activity of a template-specific nucleic acid polymerase having a 3′→5′ exonuclease activity associated with some DNA polymerases, in which one or more nucleotides are removed from the 3′ end of an oligonucleotide in a sequential manner. Polymerase enzymes having high fidelity 3′→5′ exonuclease activity are useful, for example, when primer extension must be performed with high specificity. Polymerase enzymes having 3′→5′ exonuclease proofreading activity are known to those in the art. Examples of suitable proofreading enzymes include TaKaRa LA Taq (Takara Shuzo Co., Ltd.) and Pfu (Stratagene), Vent, Deep Vent (New England Biolabs). Exemplary methods for performing long range PCR are disclosed, for example, in U.S. Pat. No. 5,436,149; Barnes, Proc. Natl. Acad. Sci. USA 91:2216-2220 (1994); Tellier et al., Methods in Molecular Biology, Vol. 226, PCR Protocols, 2nd Edition, pp. 173-177; and, Cheng et al., Proc. Natl. Acad. Sci. 91:5695-5699 (1994); the contents of which are incorporated herein by reference. In various embodiments, long range PCR involves one DNA polymerase. In some embodiments, long range PCR may involve more than one DNA polymerase. When using a combination of polymerases in long range PCR, it is possible to include one polymerase having 3′→5′ exonuclease activity, which assures high fidelity generation of the PCR product from the DNA template. Typically, a non-proofreading polymerase, which is the main polymerase is also used in conjunction with the proofreading polymerase in long range PCR reactions. Long range PCR can also be performed using commercially available kits, such as LA PCR kit available from Takara Bio Inc.

### Hybridization

There are a variety of ways by which one can assess genetic profiles, and many of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, in certain embodiments between 17 and 100 nucleotides in length up to 1-2 kilobases or more in length will allow the formation of a duplex molecule that is both stable and selective. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions.

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present disclosure in combination with an appropriate means, such as a label, for determining hybridization. A wide variety of appropriate indicator means are known in the art, including fluorescent, radioactive, enzymatic or other ligands, such as avidin/biotin, which are capable of being detected. In certain embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present disclosure are disclosed in U.S. Pat. Nos. 5,849,481, 5,849,486 and 5,851,772. The relevant portions of these and other references identified in this section of the Specification are incorporated herein by reference.

### Kits

The present compositions may be assembled into kits or pharmaceutical systems for use in detecting or diagnosing cancer associated with an RASGRF fusion and/or for use in identification of a subject responsive to a therapy directed against RASGRF or a pathway activated by RASGRF. Materials and reagents required for measuring the level of a fused RASGRF gene (i.e. a fused RASGRF polynucleotide sequence) in a sample may be assembled together in a kit. In some embodiments, the RASGRF is RASGRF1. This generally will comprise a capture reagent, primer, or probe designed to hybridize specifically to target polynucleotides of interest. The primer or probe may be labeled with a radioisotope, a fluorophore, a chromophore, a dye, an enzyme, or TOF carrier. In some embodiments, the kit comprises materials and reagents required for measuring an increased level of RASGRF expression compared to a control. In some embodiments, the kit comprises a control. Also included may be enzymes suitable for amplifying nucleic acids, including various polymerases (reverse transcriptase, Taq, etc.), dNTPs/rNTPs and buffers (e.g., 10× buffer=100 mM Tris-HCl (pH 8.3), and 500 mM KCl) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products. The kits of the disclosure may also comprise associated instructions for using the agents of the disclosure. Additionally, one or more agents for treating a cancer associated with an RASGRF fusion may be included.

Kits according to this aspect of the disclosure comprise a carrier means, such as a box, carton, tube or the like, having in close confinement therein one or more container means, such as vials, tubes, ampoules, bottles and the like. The container means of the kits will generally include at least one vial, test tube, flask, bottle, or other container means, into which a component may be placed, and in certain embodiments, suitably aliquoted. Where there is more than one component in the kit, the kit also will generally contain additional containers into which the additional components may be separately placed. However, various combinations of components may be comprised in a container. The kits of the present disclosure also will typically include a means for packaging the component containers in close confinement for commercial sale. Such packaging may include injection or blow-molded plastic containers into which the desired component containers are retained.

### In General

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook et al., 2012, volumes 1-4, Cold Spring Harbor Laboratory Press, NY); “Oligonucleotide Synthesis” (M. J. Gait, ed., Oxford University Press, 1984); “Animal Cell Culture” (R. I. Freshney, Wiley-Blackwell, 2010); “Methods in Enzymology” (S. P. Colowick, N. O. Kaplan, et al., eds., volumes 1-650, Academic Press); “Handbook of Experimental Immunology” (D. M. Weir et al., Wiley, 1997); “Gene Transfer Vectors for Mammalian Cells” (J. Miller and M. P. Calos, Cold Spring Harbor Laboratory Press, NY, 1987); “Current Protocols in Molecular Biology” (F. M. Ausubel et al., eds., John Wiley & Sons, 2002); “Polymerase Chain Reaction: Principles, Applications and Troubleshooting”, (M. E. Babar, VDM Verlag Dr. Muller, 2011); “Current Protocols in Immunology” (J. E. Coligan et al., eds., John Wiley & Sons, 1991). These techniques are applicable to the production of the polynucleotides and polypeptides of the disclosure, and, as such, may be considered in making and practicing the disclosure. Particularly useful techniques for particular embodiments will be discussed in the sections that follow.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples, therefore, specifically point out non-limiting embodiments of the present disclosure, and are not to be construed as limiting in any way the remainder of the disclosure.

The materials and methods employed in the examples are now described.

Study Participants, Sample Preparation, and Tumor Molecular Analysis. Eligible study participants provided informed written consent under an IRB-approved research protocol (Yale HIC #1010007459). Subjects were enrolled between 2011 and 2019. Tumor molecular studies obtained as part of routine clinical care were performed by the Tumor Profiling Laboratory at Yale-New Haven Hospital. Testing approaches included hotspot mutation detection via polymerase chain reaction, fluorescence in situ hybridization (FISH) to identify gene rearrangements, or the Oncomine Comprehensive Assay next generation sequencing platform to identify mutations and gene rearrangements involving 161 cancer-related genes.

25 tumors were subjected to whole-exome sequencing and RNAseq. Tumor samples were immediately stored in RNAlater Solution (ThermoFisher Scientific) and flash frozen at the time of surgical resection or biopsy. Peripheral blood and/or normal tissue were also collected from study participants for use as a germline control. Genomic DNA and RNA was isolated using the AllPrep DNA/RNA Mini Kit (Qiagen) according to the manufacturer's instructions. For tumors without available fresh frozen samples, formalin-fixed paraffin-embedded (FFPE) slides were reviewed by a pathologist to select areas enriched for tumor. Tissue cores were generated, and nucleic acids were extracted using the AllPrep DNA/RNA FFPE Kit (Qiagen) according to the manufacturer's instructions.

Whole-Exome Sequencing and RNAseq, One microgram of genomic DNA was sheared to a mean fragment length of 140 base pairs with subsequent addition of custom adapters (Integrated DNA Technologies). Adapter-ligated DNA fragments were then PCR amplified using custom-made primers with introduction of a unique 6 base index at one end of each DNA fragment. Exome capture was performed using biotinylated DNA probes (Nimblegen, SeqCap EZ Exome version 2, part #05860504001) according to the manufacturer's instructions. Samples were sequenced using 150 bp paired end sequencing on an Illumina NovaSeq according to the manufacturer's protocols to a mean coverage of 150× for tumor and 75× for matched germline control. For RNA samples, the quality of total RNA was verified using a Bioanalyzer (Agilent). mRNA was isolated from total RNA using the mRNA-seq Sample Prep Kit (Illumina, Cat #1004814). Samples were sequenced using 150 bp paired end sequencing on an Illumina NovaSeq at 50 million reads per sample.

Sequencing reads from exome-captured samples were analyzed with a combination of germline and somatic variant calling. BAM files of aligned reads were created for each sample by aligning the sequencing reads to the GRCh37 human reference with decoy sequences (the “hs37d5” reference) using BWA MEM v0.7.15, marking duplicates using Picard MarkDuplicates v2.17.11, and then performing indel realignment and base quality score recalibration using GATK v3.4. Then, variants were called using the tumor/normal bam files in three ways: (1) a joint variant call using GATK HaplotypeCaller, GenotypeGVCFs and hard filtering following GATK 3.4 best practices; (2) somatic SNP variant calls using MuTect v2.7.1; (3) somatic indel variant calls using Indelocator v33.3336. The output from the three variant callers were merged using in-house scripts into a single VCF file, containing the union of GATK variants and MuTect/Indelocator somatic variants. Those variants were annotated using Annovar and VEP.

Copy-number variant (CNV) regions were identified by calculating the mean read depth for each RefGene coding exon for the tumor and normal samples. Normalized tumor/normal read depth ratios were computed for each exon (normalized by the mean read depth of the tumor and normal across the exome), and a mean ratio for each 20 kb region of the genome containing an exon was computed. Those mean ratios were de-noised and segmented by circular binary segmentation using the DNAcopy library from R. Regions with a value deviating from the expected 1.0 ratio were identified as CNVs. For each CNV, ploidy is calculated using a deviation step value of 0.5 for high tumor purity samples (purity>80%, a 0.4 step for purity between 40% and 80%, and 0.32 step for purity less than 40%), with the ploidy equaling 2 plus or minus the number of deviation steps.

For RNAseq, reads were mapped to the human reference genome (hg19) using HISAT2 (v2.1.0). Gene expression levels were quantified using StringTie (v1.3.3b) with gene models (v27) from the GENCODE project. Differentially expressed genes were identified using DESeq2 (v1.22.1). Fusion genes were identified using FusionCatcher (v1.00). Gene fusion events and exon-skipping events were manually inspected by loading BAM files into the Integrative Genomics Viewer (IGV).

Cloning of Full-Length OCLN-RASGRF1, SLC4A4-RASGRF1, and IQGAP1-RASGRF1 cDNA. PaCaDD137 cells were purchased from DSMZ (Germany) and cultured in an 80% mixture of DMEM and Keratinocyte SFM (at 1:1), 20% heat-inactivated fetal bovine serum (Gibco), and penicillin (100 units/mL)/streptomycin (100 μg/mL; Gibco). Total RNA from YLCB Tumor 9 (for OCLN-RASGRF1) or PaCaDD137 (for SLC4A4-RASGRF1) was used as a template for cDNA preparation with oligo-dT using the SuperScript III First-Strand Synthesis System (Invitrogen). Full-length fusion transcripts were amplified using the Q5 High-Fidelity DNA Polymerase (New England BioLabs). PCR primers were designed from the 5′ end of OCLN and SLC4A4 and 3′ end of RASGRF1. For OCLN-RASGRF1, a primary PCR was performed using forward primer CATCCTGAAGATCAGCTGACCATTG (SEQ ID NO: 1) and reverse primer CGAATATGTACAGTATCATCTAGCACATGTCC (SEQ ID NO: 2). A secondary PCR was then performed using nested PCR primers with incorporation of attB recombination sites for Gateway cloning (Invitrogen). Sequences for these primers were GGGGACAACTTTGTACAAAAAAGTTGGCACCCCGCCATGTCATCCAGGCCTCTT GAAAGTCCAC (SEQ ID NO: 3; forward) and GGGGACAACTTT GTACAAGAAAGTTGGCAA TCAGGTGGGGAGTTTTGGTTCTATTCG (SEQ ID NO: 4; reverse). For amplification of SLC4A4-RASGRF1, PCR was performed using a forward PCR primer specific to SLC4A4 with incorporation of an attB recombination site (GGGGACAACTTTGTACAAAAAAGTTGGCAC CATGGAGGATGAAGCTGTCCTGGACAG) (SEQ ID NO: 5) and the same reverse primer with an attB site used for OCLN-RASGRF1.

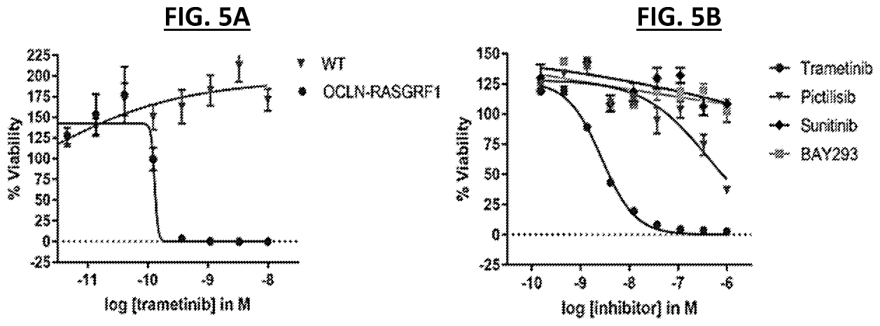

For IQGAP1-RASGRF1, a primary PCR was performed using the forward primer indicated in Supplementary FIG. 5A and the same reverse primer with an attB site above. A secondary PCR was then performed with a forward PCR primer with incorporation of an attB site (GGGGACAACTTTGTACAAAAAAGTTGGCACCATGTCCGCCGCAGACGAGGTTG ACGGG) (SEQ ID NO: 6) and the same reverse primer used for the primary PCR.

PCR products were gel-purified and cloned into pDONR223 (Invitrogen) using BP Clonase (Invitrogen). OCLN-RASGRF1, SLC4A4-RASGRF1, and IQGAP1-RASGRF1 cDNAs were then recombined into the lentiviral expression vector pLX307 (Addgene) using LR Clonase (Invitrogen). pLX307 directs mammalian expression from the E1Fa promoter and contains a puromycin selectable marker. The entire coding sequences of OCLN-RASGRF1 and SLC4A4-RASGRF1 cDNAs were confirmed by Sanger sequencing. Lentivirus was produced using BEK 293T cells as previously described.

Antibodies and Immunoblotting. PaCaDD137 cells were purchased from DSMZ (Germany) and cultured in an 80% mixture of DMEM and Keratinocyte SFM (at 1:1), 20% heat-inactivated fetal bovine serum (Gibco), and penicillin (100 units/mL)/streptomycin (100 μg/mL; Gibco). Cells were lysed in RIPA buffer (Abcam) supplemented with phosphatase inhibitors (Sigma) and protease inhibitors (Roche). Lysates were fractionated by SDS-polyacrylamide gel electrophoresis and transferred to nitrocellulose membranes using the Trans-Blot Turbo transfer system (Bio-Rad). Chemiluminescence was performed using SuperSignal West Pico Chemiluminescent substrate (Thermo Scientific). Antibody against RASGRF1 (ab 111830) was obtained from Abcam. Antibodies against AKT (#29290), phopho-AKT (Ser 473; #4060), ERK1/2 (#4695), phopho-ERK1/2 (Thr 202/Tyr 204, #9106), cleaved mouse PARP (#9548), and actin (#3700) were obtained from Cell Signaling. Anti-vinculin (#V9131) was obtained from Sigma.

RAS Activation Assay. HEK 293T cells were transduced with OCLN-RASGRF1, SLC4A4-RASGRF1, or green fluorescent protein (GFP) in pLX307. Following puromycin selection, cell lysates were collected and subjected to an RAS activation assay using the Active RAS Detection kit (Cell Signaling Cat #8821). Briefly, cell lysates were incubated with glutathione resin and GST-Raf1-RBD according to the manufacturer's instructions for affinity precipitation of GTP-bound RAS. Unbound proteins were cleared by centrifugation, and eluted samples containing GTP-bound RAS were subjected to Western immunoblotting using the provided RAS mouse antibody.

Cell Surface Protein Biotinylation and Purification. Cell surface protein biotinylation and purification were performed using the Pierce Cell Surface Protein Isolation kit (ThermoFisher Cat. #89881) according to the manufacturer's instructions. Briefly, HEK 293T cells with ectopic expression of OCLN-RASGRF1 and PaCaDD137 cells expressing endogenous SLC4A4-RASGRF1 (3-4 ten cm dishes each) were incubated with the biotinylation reagent for 30 minutes on ice (for HEK 293T cells) or 10 minutes at room temperature (for PaCaDD137 cells). Following cell lysis, labeled surface proteins were affinity purified using NeutrAvidin agarose resin and eluted as per the manufacturer's instructions.

Anchorage-Independent Transformation Assays. NIH3T3 cells were maintained in RPMI 1640 (Gibco) with 10% heat-inactivated fetal bovine serum (Gibco) and penicillin (100 units/mL)/streptomycin (100 μg/mL; Gibco). NIH3T3 cells were transduced with OCLN-RASGRF1, SLC4A4-RASGRF1, green fluorescent protein (GFP), or EML4-ALK in pLX307 in the presence of 4 μg/mL polybrene. Cells were centrifuged at 2250 rpm for 30 minutes. 24 hours later, transduced cells were selected with 0.75 μg/mL puromycin for at least 5 days. After selection, 40,000 cells were plated in triplicate in growth media containing 0.4% agar on top of a layer of growth media in 0.8% agar in a 6-well tray. Cells were cultured in agar for 14 to 19 days with fresh media added weekly. Images were captured using a Zeiss Observer A1 microscope using a 5× objective. Quantification of colony area was performed using ImageJ software.

Ba F3 Transformation Assays. Ba/F3 cells were maintained in RPMI 1640 (Gibco) with heat-inactivated 10% fetal bovine serum (Gibco) and penicillin (100 units/mL)/streptomycin (100 μg/mL; Gibco) and 1 ng/mL interleukin-3 (IL-3; Gibco). cDNAs (GFP or OCLN-RASGRF1) in pLX307 were introduced into Ba/F3 cells via lentiviral transduction with 8 μg/mL polybrene and centrifugation at 2250 rpm at 37 degrees C. for 30 minutes. 24 hours later, transduced cells were selected with 1 μg/mL puromycin for at least 5 days. IL-3 concentrations were then reduced stepwise from 1 ng/mL to 0.01, 0.005, 0.001, and 0 ng/mL IL-3 over approximately 2-3 weeks (until IL-3-independent growth was observed for cells expressing OCLN-RASGRF1).

Cell Viability Assays and RASGRF1 knockdown. Ba/F3 cells or PaCaDD137 cells were seeded in 96-well microtiter plates. Drug or drug vehicle was added on the same day (for Ba/F3 cells) or 24 hours after seeding (for PaCaDD137 cells). Following 4-5 days of drug exposure, cell viability was determined using the Cell Titer-Glo luminescent assay (Promega) according to the manufacturer's instructions. Luminescence was measured with a Spectramax M3 plate reader (Molecular Devices). Data analysis was performed using GraphPad Prism software.

siRNA reagents targeting RASGRF1 (siRNA ID #SASI_Hs01_00211038, SASI_Hs01_00211039, SASI_Hs01_00211040) or negative control (MISSION siRNA Universal Negative Control #1, Cat. #SIC001) were purchased from Sigma. PaCaDD137 cells were transfected with 50 nM siRNA using Lipofectamine RNAiMAX transfection reagent (ThermoFisher Cat. #13778075) according to the manufacturer's instructions for reverse transfection in a 24-well format for Western immunoblotting or a 96-well format for cell viability assays. For Western immunoblotting, lysates were prepared 72 hours after transfection. For cell viability assays, viability was determined using Cell Titer-Glo 7 days after transfection.

Xenograft Drug Dosing Assays. 5×10⁶ PaCaDD137 cells were subcutaneously implanted into the right flank of 8-week-old female immune-deficient Rag2/TL2RG double knockout mice (Envigo) in the presence of Matrigel (Corning). Cells were permitted to engraft for one week until tumors reached ˜100 mm³ in volume. Mice were then randomized to receive 1 mg/kg/day trametinib (n=10) or vehicle (6.25% DMSO in PBS; n=10). A stock solution of 4 mg/mL trametinib (Cayman Chemical) was formulated in DMSO and diluted to 0.25 mg/mL in PBS. An equivalent volume of DMSO was dissolved in PBS for vehicle. Trametinib or vehicle was administered daily by intraperitoneal injection in a volume of 100 μL. Tumor dimensions were recorded by caliper measurements every 3 days [0.5×(length)×(width)²]. The experiment was terminated and mice were euthanized when any tumor reached a volume of 1000 mm³. Experiments were conducted in accordance with Yale Institutional Animal Care and Use Committee guidelines under an approved research protocol.

### Example 1: Evaluation of Established Oncogenic Drivers in NSCLC from Never or Light Smokers

Since its inception in 2011, the Yale Lung Cancer Biorepository (YLCB) has collected tumor specimens and peripheral blood from nearly 1000 individuals diagnosed with NSCLC under an IRB-approved research protocol. This effort focuses on newly-diagnosed patients presenting to Yale-New Haven Hospital with early-stage disease amenable to surgical resection. In addition to banking of biological material, clinical and epidemiologic annotation of samples is prioritized to facilitate correlative studies.

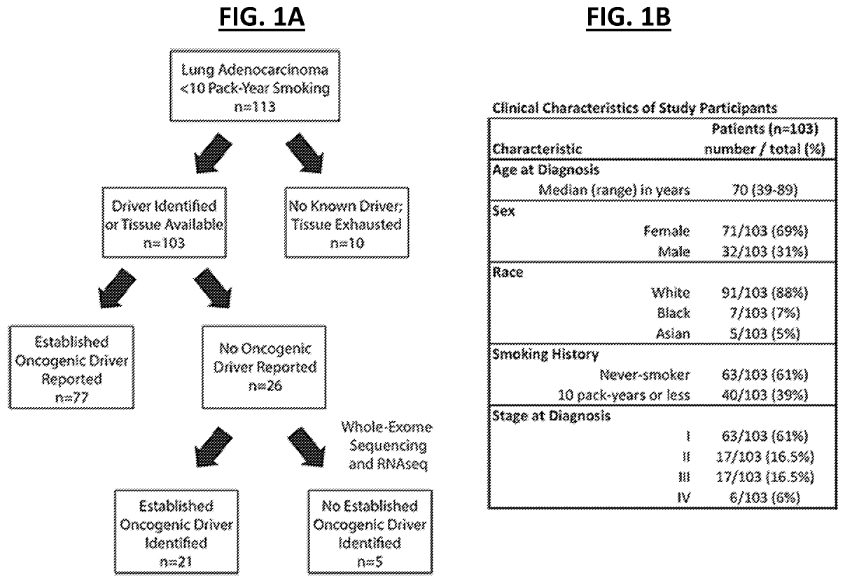

Within the YLCB, lung adenocarcinomas obtained from 113 individuals who were either never-smokers or had no more than a 10 pack-year smoking history were identified (FIG. 1A). Tissue blocks had been previously exhausted without identification of an established oncogenic driver for 10 of these tumors. For the remaining 103 tumors, either an oncogenic driver had previously been identified or there was archival tumor available for molecular profiling. Clinical characteristics associated with these 103 tumors are shown in FIG. 1B. Age at diagnosis ranged from 39 to 89 with a median of 70 years. 71 tumors were identified from women and 32 from men. Racial background of participants included Caucasian (91), African American (7), and Asian (5). 63 individuals had never smoked. The majority of the 103 tumors were early-stage diagnoses (63 Stage I, 17 Stage II, 17 Stage III, and 6 Stage IV).

78 of the 103 tumors harbored an established oncogenic driver identified by tumor molecular profiling obtained during routine clinical care. Drivers were identified by the Yale Tumor Profiling Laboratory at Yale-New Haven Hospital using various approaches. These include hotspot mutation detection via polymerase chain reaction, FISH (to identify ALK, ROS1, or RET gene rearrangements), or the Oncomine Comprehensive Assay next generation sequencing platform to identify mutations and gene rearrangements involving 161 cancer-related genes. Identified molecular drivers from these 78 tumors included activating alterations in EGFR, KRAS, ALK, ERBB2, RET, and BRAF.

An established oncogenic driver was not reported in the remaining 25 tumors, although a subset of these tumors had been subjected to only limited or no molecular analysis (FIG. 1A). To further characterize these tumors, DNA and RNA was isolated from these specimens and whole-exome sequencing (WES) and RNAseq were performed. Genomic DNA extracted from matched peripheral blood was subjected to WES as a germline control for each tumor. Of the 25 tumors, an established oncogenic driver was identified in 20. These included activating alterations in EGFR, KRAS, ERBB2, PIK3CA, copy number gain of ERBB2, RET rearrangements, and MET Exon 14 splice alterations.

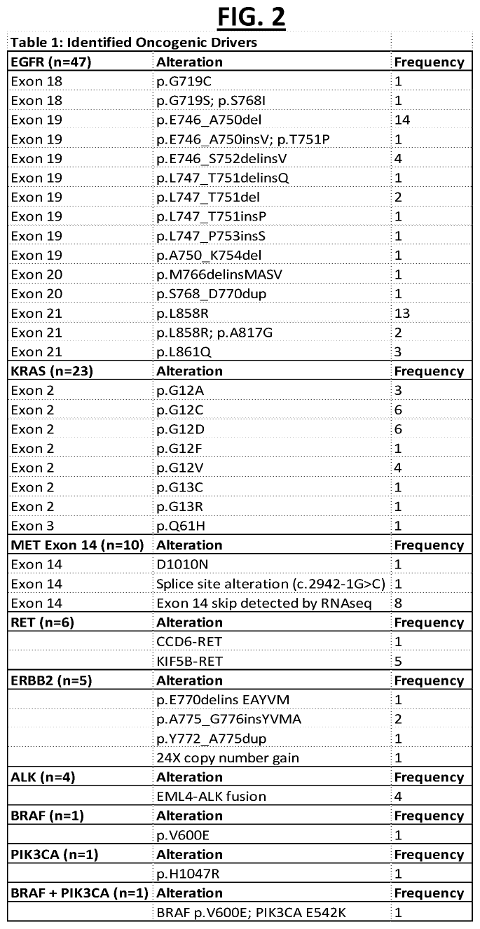

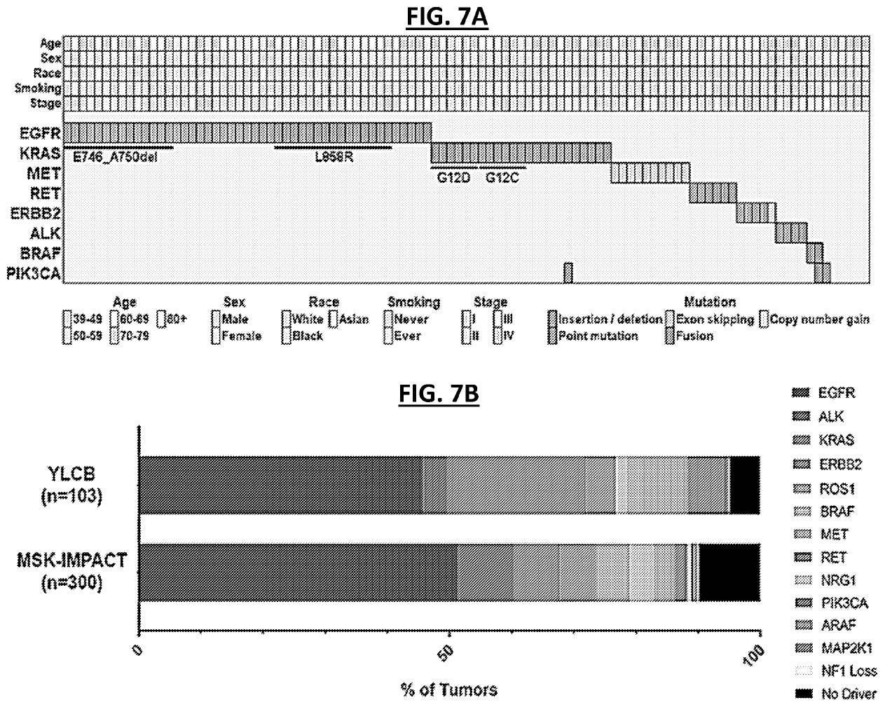

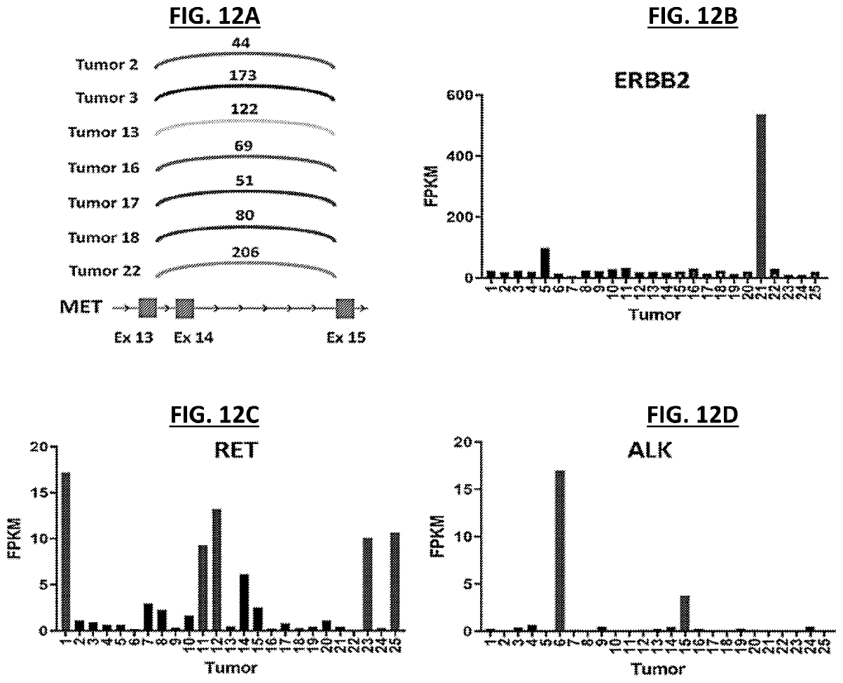

In total, an oncogenic driver was identified in 98 of 103 (95%) LUADs evaluated (FIG. 2 and FIGS. 7A-7B). Consistent with prior reports, nearly half of the tumors harbored activating EGFR alterations (n=47). Among these were 25 tumors with Exon 19 deletion, 15 with L858R, and 2 with Exon 20 insertions. Activating KRAS alterations were observed in 23 tumors including 6 with a KRAS G12C alteration. One tumor with a KRAS G12A alteration had a concurrent PIK3CA H1047R mutation. MET Exon 14 splice alterations were observed in 10 tumors (FIG. 12A). ERBB2 alterations were identified in 5 tumors, including 4 with activating mutations and 1 with marked copy number gain (24× ploidy and 22.9-fold increased expression by RNA-seq; FIG. 12B). Activating RET (n=6) and ALK (n=4) rearrangements were associated with increased RET and ALK expression, respectively (FIGS. 12C-12D). BRAF V600E was identified in 2 tumors, including one tumor with a concurrent PIK3CA E542K alteration. An activating PIK3CA H1047R mutation was identified in one tumor. Activating alterations in other established oncogenic drivers (including ROS1, TRK, NRAS, NRG1, RIT1, ARAF, and MAP2K1) were not identified in this cohort.

The prevalence of oncogenic drivers was then confirmed in an independent dataset. Using the CBioPortal for Cancer Genomics, data from the MSK-IMPACT Clinical Sequencing Cohort was queried to identify lung adenocarcinomas from never-smokers that had undergone hybridization capture-based next-generation sequencing of up to 410 cancer-associated genes using the MSK-IMPACT platform. In contrast to the cohort studied herein (which consisted primarily of treatment-naïve early-stage NSCLC), most tumors profiled with MSK-IMPACT represent advanced and often heavily treated disease. Of 300 profiled lung adenocarcinomas from never-smokers in the database, it was found that 271 tumors (90%) harbored established activating alterations in EGFR, KRAS, ERBB2, MET, ALK, ROS1, RET, BRAF, NRG1, and NTRK1/2/3 (FIG. 7D). Together, these combined findings suggest a known driver oncogene can be identified from approximately 90% or more of lung adenocarcinomas from individuals with <10 pack-year smoking history.

### Example 2: Identification of OCLN-RASGRF1 and SLC4A4-RASGRF1 Fusions

An established oncogenic driver was not identified in 5 tumors characterized from the YLCB (FIGS. 1A, 7A).). Four of these tumors were obtained from never-smokers, while one was from a patient with an 8 pack-year smoking history (FIG. 7A). The whole-exome and RNA sequencing data from these tumors was examined in search of previously uncharacterized candidate driver events. One of these tumors, YLCB345, was a Stage I lung adenocarcinoma obtained from a 59 year-old Caucasian female never-smoker. This patient presented with an incidental finding of a right lower lobe lung nodule that increased in size to 2 cm on subsequent imaging and was ultimately resected by lobectomy. Histologic subtype was acinar-predominant (85%) with minor components of lepidic (10%) and papillary with mucinous features (5%). Adjuvant therapy was not indicated, and to date the patient has not experienced cancer recurrence.

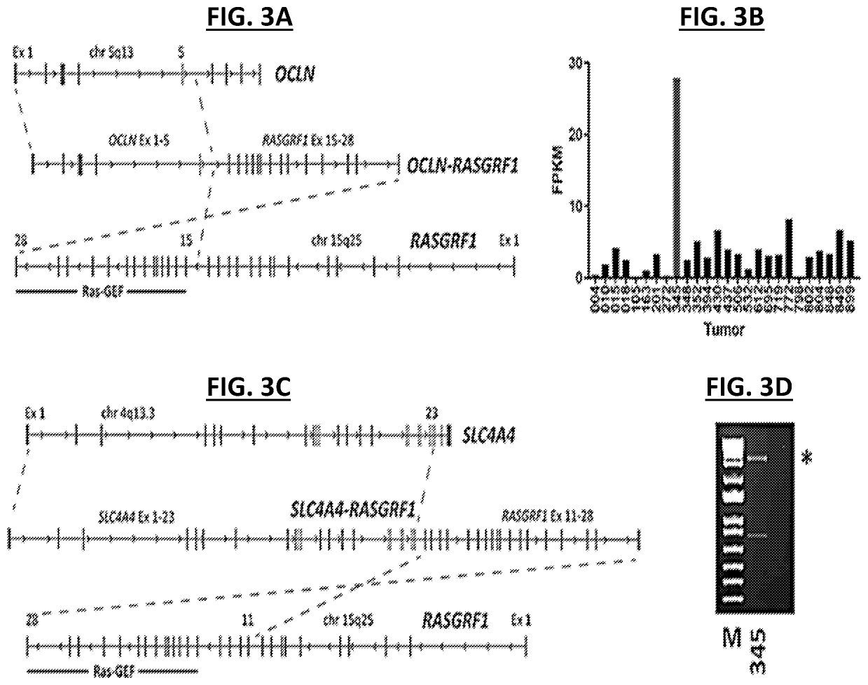

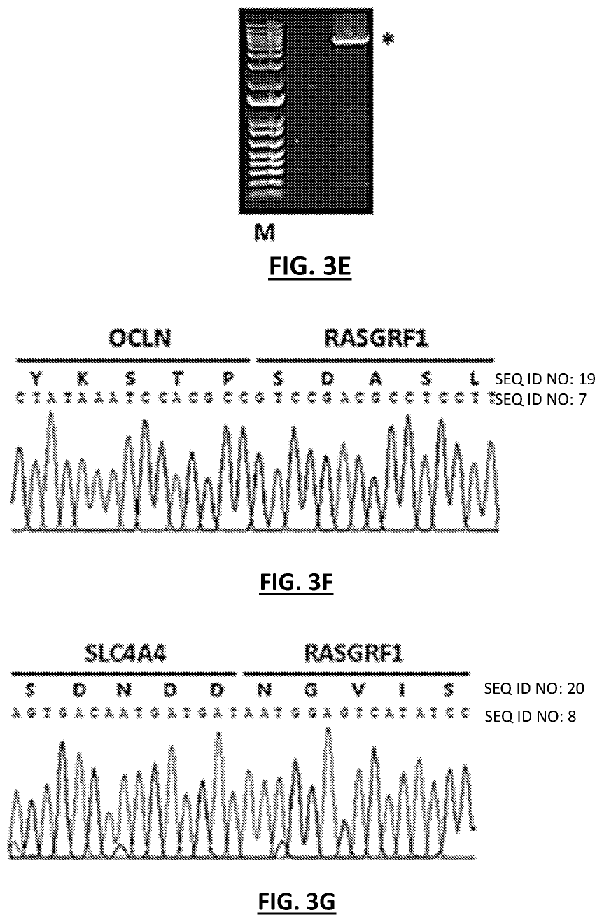

From RNAseq of Tumor 9, a novel gene rearrangement predicted to encode an N-terminal segment of the tight junction transmembrane protein occludin (OCLN) fused in-frame with a C-terminal segment of the guanine exchange factor (GEF) RASGRF1 was identified (FIG. 3A). 26 unique reads were identified spanning the fusion, which occurred between the 3′ end of Exon 5 of OCLN and the 5′ end of Exon 15 of RASGRF. Exons 1-5 of OCLN include all 4 transmembrane domains of the protein product but lack the carboxy-terminal cytoplasmic tail. The carboxy-terminal Ras-GEF domain of RASGRF1 catalyzes the dissociation of GDP from RAS family proteins, promoting RAS activation via transition from inactive GDP-bound RAS to activated GTP-bound RAS. The entire Ras-GEF domain of RASGRF1 is contained within the segment included in the OCLN-RASGRF1 fusion protein and has previously been shown to have catalytic activity in the absence of the N-terminal half of RASGRF1 (Chevallier-Multon, M. C. et al., J. Biol. Chem., 1993, 268:11113-11118). Furthermore, overexpression of wild-type RASGRF1 promotes transformation and anchorage-independent growth in mouse fibroblasts (Chevallier-Multon, M. C. et al., J. Biol. Chem., 1993, 268:11113-11118).

In normal human tissues, OCLN is highly expressed in thyroid and lung while RASGRF1 is expressed primarily in brain (with lesser expression in lung) (Fagerberg, L. et al., Mol. Cell Proteomics, 13:397-406). It was reasoned that OCLN-RASGRF1 could lead to increased expression of the C-terminal Ras-GEF domain under the OCLN promoter. To assess this, RASGRF1 expression (using reads mapping to the C-terminal segment included in OCLN-RASGRF1) was compared across all 26 lung adenocarcinomas for which we had performed RNAseq. Of these tumors, expression of RASGRF1 in YLCB345 was 8.7-fold higher than the mean RASGRF1 expression in the other 25 tumors (FIG. 3B).

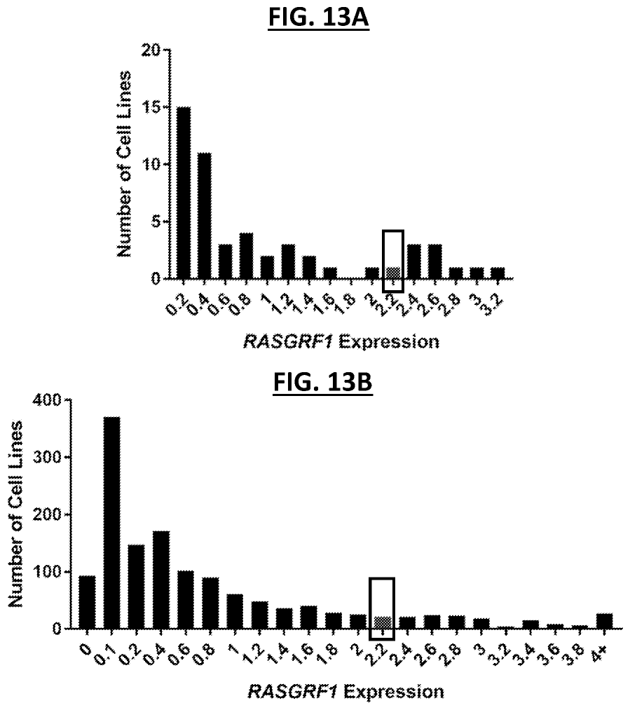

Next, the Cancer Dependency Map portal was used to search for similar fusions in sequenced cancer cell lines. A cell line (PaCaDD137) derived from a 75 year-old Caucasian female with an early-stage pancreatic ductal adenocarcinoma was identified harboring a similar gene rearrangement fusing Exons 1-23 of the transmembrane protein SLC4A4 in-frame with Exons 11-28 of RASGRF1 (FIG. 3C). SLC4A4 encodes a sodium bicarbonate cotransporter that is highly expressed in kidney and pancreas, raising the possibility that this gene rearrangement could lead to increased expression of the C-terminal RAS-GEF domain in pancreatic tumor cells under the SLC4A4 promoter (Fagerberg, L. et al., Mol. Cell Proteomics, 13:397-406). Of note, PaCaDD137 cells lack an activating KRAS mutation (present in approximately 90% of PDACs). Using RNA-seq data from the Cancer Cell Line Encyclopedia (CCLE), we examined RASGRF1 expression across 1378 cancer cell lines (including PaCaDD137 and 51 additional PDAC cell lines)¹⁵. RASGRF1 expression was higher in PaCaDD137 cells than in 82% of PDAC cell lines and 88% of total CCLE lines (FIG. 13).

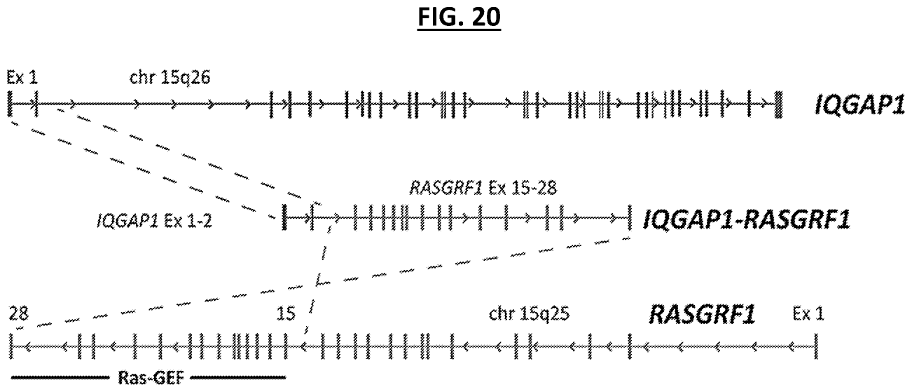

A search of the Tumor Fusion Gene Data Portal (tumorfusions.org) for RASGRF1 fusions was then performed. Fusion genes in the portal are identified using RNA-seq data generated from tumors in The Cancer Genome Atlas (TCGA). The search found a giant cell sarcoma of the retroperitoneum obtained from an 87 year-old Caucasian male (ID #TCGA-FX-A3TO) harboring an in-frame gene fusion between Exons 1-2 of IQGAP1 and Exons 15-28 of RASGRF1 (FIG. 20). IQGAP1 is ubiquitously expressed and encodes a scaffolding protein with roles in cell-cell adhesion, cytoskeletal regulation, and other biological processes. Of note, only the first 52 of 1657 amino acids of IQGAP1 are predicted to be included in the IQGAP1-RASGRF1 fusion protein.

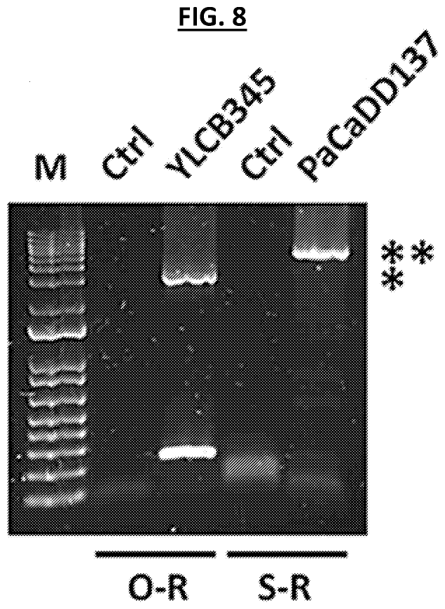

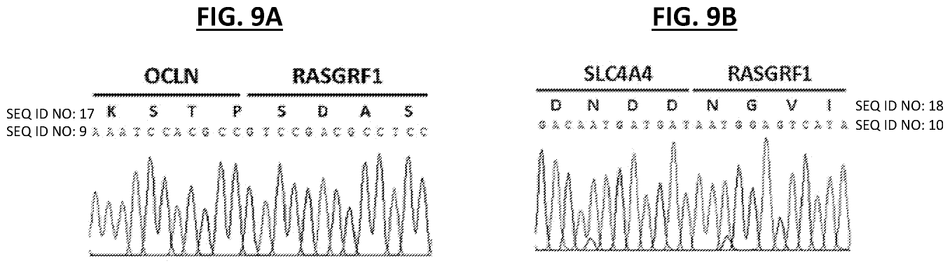

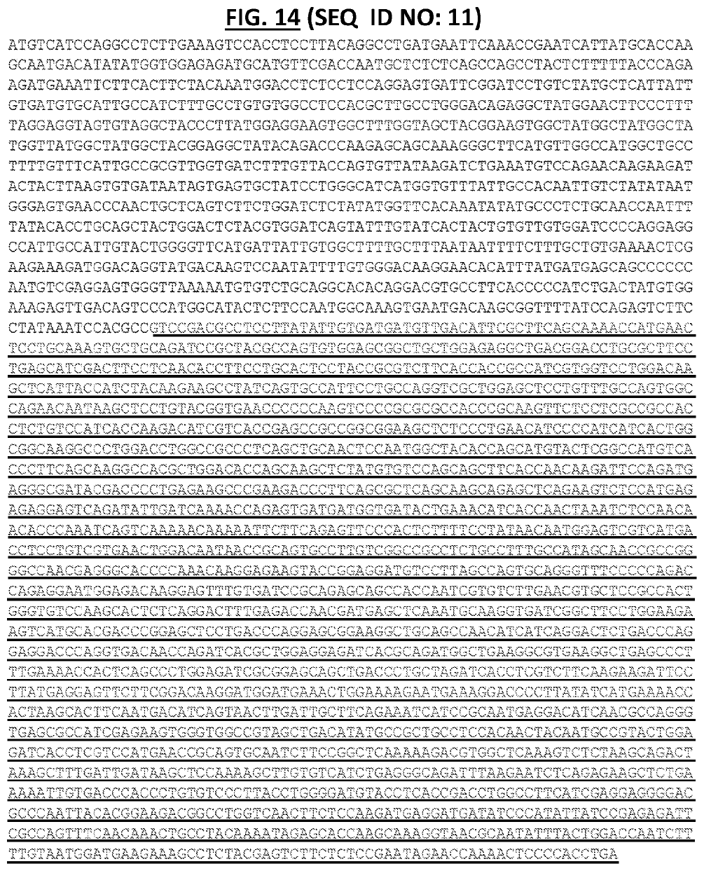

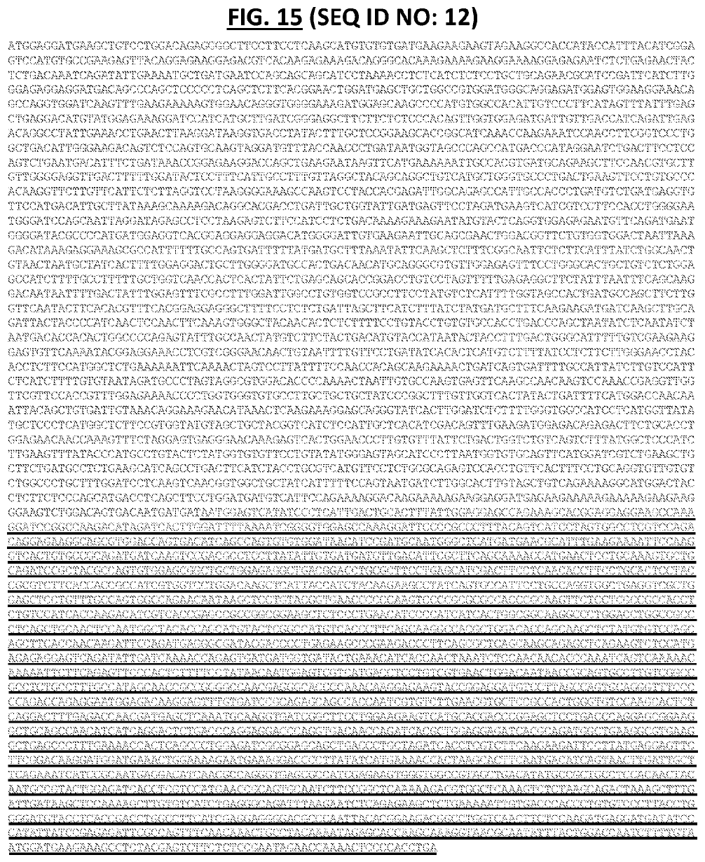

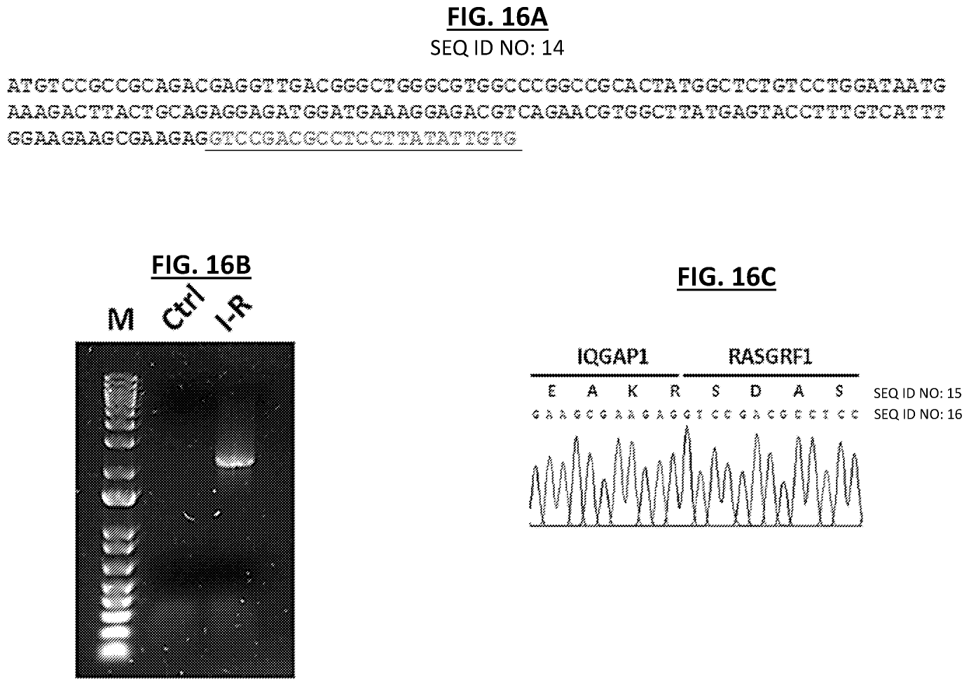

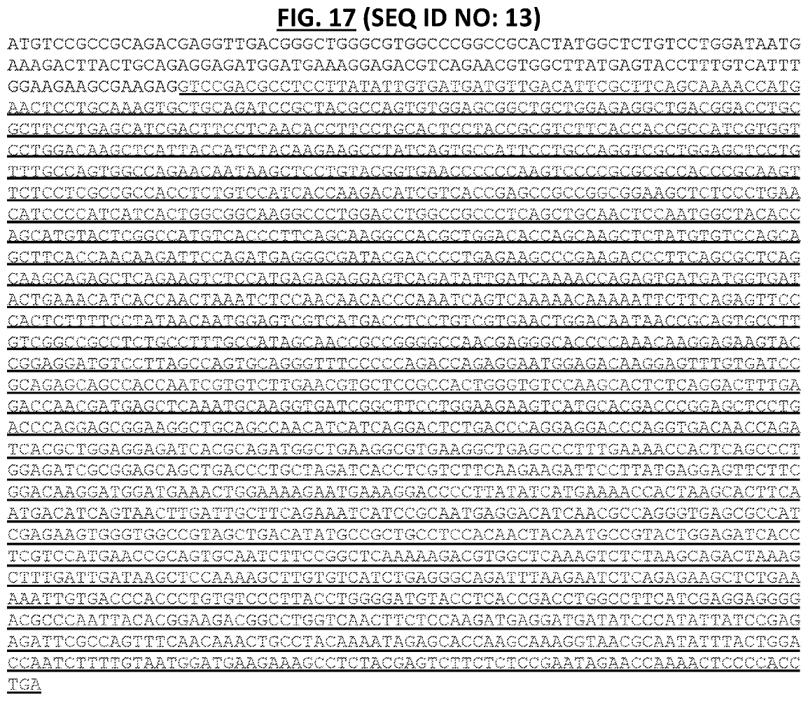

cDNA was then separately generated from RNA obtained from YLCB345 and from PaCaDD137 cells. PCR amplification of the full-length 3 kb OCLN-RASGRF1 and 5.3 kb SLC4A4-RASGRF1 fusion transcripts was performed from cDNA (FIGS. 3D-3E and FIG. 8). The in-frame fusions joining Exons 1-5 of OCLN with Exons 15-28 of RASGRF1 and Exons 1-23 of SLC4A4 with Exons 11-28 of RASGRF1 were confirmed by Sanger sequencing (FIGS. 3F-3G and FIGS. 9A-9B and FIGS. 14 and 15). A full-length 2.1 kb IQGAP1-RASGRF1 cDNA was also derived using PCR with OCLN-RASGRF1 as template and a forward primer incorporating the first two exons of IQGAP1 (FIGS. 16 and 17).

OCLN-RASGRF1 Fusion RASGRF1 sequence is underlined
(SEQ ID NO: 11)
ATGTCATCCAGGCCTCTTGAAAGTCCACCTCCTTACAGGCCTGATGAATTCAAACCGAATCA
TTATGCACCAAGCAATGACATATATGGTGGAGAGATGCATGTTCGACCAATGCTCTCTCAGC
CAGCCTACTCTTTTTACCCAGAAGATGAAATTCTTCACTTCTACAAATGGACCTCTCCTCCA
GGAGTGATTCGGATCCTGTCTATGCTCATTATTGTGATGTGCATTGCCATCTTTGCCTGTGT
GGCCTCCACGCTTGCCTGGGACAGAGGCTATGGAACTTCCCTTTTAGGAGGTAGTGTAGGCT
ACCCTTATGGAGGAAGTGGCTTTGGTAGCTACGGAAGTGGCTATGGCTATGGCTATGGTTAT
GGCTATGGCTACGGAGGCTATACAGACCCAAGAGCAGCAAAGGGCTTCATGTTGGCCATGGC
TGCCTTTTGTTTCATTGCCGCGTTGGTGATCTTTGTTACCAGTGTTATAAGATCTGAAATGT
CCAGAACAAGAAGATACTACTTAAGTGTGATAATAGTGAGTGCTATCCTGGGCATCATGGTG
TTTATTGCCACAATTGTCTATATAATGGGAGTGAACCCAACTGCTCAGTCTTCTGGATCTCT
ATATGGTTCACAAATATATGCCCTCTGCAACCAATTTTATACACCTGCAGCTACTGGACTCT
ACGTGGATCAGTATTTGTATCACTACTGTGTTGTGGATCCCCAGGAGGCCATTGCCATTGTA
CTGGGGTTCATGATTATTGTGGCTTTTGCTTTAATAATTTTCTTTGCTGTGAAAACTCGAAG
AAAGATGGACAGGTATGACAAGTCCAATATTTTGTGGGACAAGGAACACATTTATGATGAGC
AGCCCCCCAATGTCGAGGAGTGGGTTAAAAATGTGTCTGCAGGCACACAGGACGTGCCTTCA
CCCCCATCTGACTATGTGGAAAGAGTTGACAGTCCCATGGCATACTCTTCCAATGGCAAAGT
GAATGACAAGCGGTTTTATCCAGAGTCTTCCTATAAATCCACGCCGTCCGACGCCTCCTTAT
ATTGTGATGATGTTGACATTCGCTTCAGCAAAACCATGAACTCCTGCAAAGTGCTGCAGATC
CGCTACGCCAGTGTGGAGCGGCTGCTGGAGAGGCTGACGGACCTGCGCTTCCTGAGCATCGA
CTTCCTCAACACCTTCCTGCACTCCTACCGCGTCTTCACCACCGCCATCGTGGTCCTGGACA
AGCTCATTACCATCTACAAGAAGCCTATCAGTGCCATTCCTGCCAGGTCGCTGGAGCTCCTG
TTTGCCAGTGGCCAGAACAATAAGCTCCTGTACGGTGAACCCCCCAAGTCCCCGCGCGCCAC
CCGCAAGTTCTCCTCGCCGCCACCTCTGTCCATCACCAAGACATCGTCACCGAGCCGCCGGC
GGAAGCTCTCCCTGAACATCCCCATCATCACTGGCGGCAAGGCCCTGGACCTGGCCGCCCTC
AGCTGCAACTCCAATGGCTACACCAGCATGTACTCGGCCATGTCACCCTTCAGCAAGGCCAC
GCTGGACACCAGCAAGCTCTATGTGTCCAGCAGCTTCACCAACAAGATTCCAGATGAGGGCG
ATACGACCCCTGAGAAGCCCGAAGACCCTTCAGCGCTCAGCAAGCAGAGCTCAGAAGTCTCC
ATGAGAGAGGAGTCAGATATTGATCAAAACCAGAGTGATGATGGTGATACTGAAACATCACC
AACTAAATCTCCAACAACACCCAAATCAGTCAAAAACAAAAATTCTTCAGAGTTCCCACTCT
TTTCCTATAACAATGGAGTCGTCATGACCTCCTGTCGTGAACTGGACAATAACCGCAGTGCC
TTGTCGGCCGCCTCTGCCTTTGCCATAGCAACCGCCGGGGCCAACGAGGGCACCCCAAACAA
GGAGAAGTACCGGAGGATGTCCTTAGCCAGTGCAGGGTTTCCCCCAGACCAGAGGAATGGAG
ACAAGGAGTTTGTGATCCGCAGAGCAGCCACCAATCGTGTCTTGAACGTGCTCCGCCACTGG
GTGTCCAAGCACTCTCAGGACTTTGAGACCAACGATGAGCTCAAATGCAAGGTGATCGGCTT
CCTGGAAGAAGTCATGCACGACCCGGAGCTCCTGACCCAGGAGCGGAAGGCTGCAGCCAACA
TCATCAGGACTCTGACCCAGGAGGACCCAGGTGACAACCAGATCACGCTGGAGGAGATCACG
CAGATGGCTGAAGGCGTGAAGGCTGAGCCCTTTGAAAACCACTCAGCCCTGGAGATCGCGGA
GCAGCTGACCCTGCTAGATCACCTCGTCTTCAAGAAGATTCCTTATGAGGAGTTCTTCGGAC
AAGGATGGATGAAACTGGAAAAGAATGAAAGGACCCCTTATATCATGAAAACCACTAAGCAC
TTCAATGACATCAGTAACTTGATTGCTTCAGAAATCATCCGCAATGAGGACATCAACGCCAG
GGTGAGCGCCATCGAGAAGTGGGTGGCCGTAGCTGACATATGCCGCTGCCTCCACAACTACA
ATGCCGTACTGGAGATCACCTCGTCCATGAACCGCAGTGCAATCTTCCGGCTCAAAAAGACG
TGGCTCAAAGTCTCTAAGCAGACTAAAGCTTTGATTGATAAGCTCCAAAAGCTTGTGTCATC
TGAGGGCAGATTTAAGAATCTCAGAGAAGCTCTGAAAAATTGTGACCCACCCTGTGTCCCTT
ACCTGGGGATGTACCTCACCGACCTGGCCTTCATCGAGGAGGGGACGCCCAATTACACGGAA
GACGGCCTGGTCAACTTCTCCAAGATGAGGATGATATCCCATATTATCCGAGAGATTCGCCA
GTTTCAACAAACTGCCTACAAAATAGAGCACCAAGCAAAGGTAACGCAATATTTACTGGACC
AATCTTTTGTAATGGATGAAGAAAGCCTCTACGAGTCTTCTCTCCGAATAGAACCAAAACTC
CCCACCTGA
SLC4A4-RASGRF1 Fusion RASGRF1 sequence is underlined
(SEQ ID NO: 12)
ATGGAGGATGAAGCTGTCCTGGACAGAGGGGCTTCCTTCCTCAAGCATGTGTGTGATGAAGA
AGAAGTAGAAGGCCACCATACCATTTACATCGGAGTCCATGTGCCGAAGAGTTACAGGAGAA
GGAGACGTCACAAGAGAAAGACAGGGCACAAAGAAAAGAAGGAAAAGGAGAGAATCTCTGAG
AACTACTCTGACAAATCAGATATTGAAAATGCTGATGAATCCAGCAGCAGCATCCTAAAACC
TCTCATCTCTCCTGCTGCAGAACGCATCCGATTCATCTTGGGAGAGGAGGATGACAGCCCAG
CTCCCCCTCAGCTCTTCACGGAACTGGATGAGCTGCTGGCCGTGGATGGGCAGGAGATGGAG
TGGAAGGAAACAGCCAGGTGGATCAAGTTTGAAGAAAAAGTGGAACAGGGTGGGGAAAGATG
GAGCAAGCCCCATGTGGCCACATTGTCCCTTCATAGTTTATTTGAGCTGAGGACATGTATGG
AGAAAGGATCCATCATGCTTGATCGGGAGGCTTCTTCTCTCCCACAGTTGGTGGAGATGATT
GTTGACCATCAGATTGAGACAGGCCTATTGAAACCTGAACTTAAGGATAAGGTGACCTATAC
TTTGCTCCGGAAGCACCGGCATCAAACCAAGAAATCCAACCTTCGGTCCCTGGCTGACATTG
GGAAGACAGTCTCCAGTGCAAGTAGGATGTTTACCAACCCTGATAATGGTAGCCCAGCCATG
ACCCATAGGAATCTGACTTCCTCCAGTCTGAATGACATTTCTGATAAACCGGAGAAGGACCA
GCTGAAGAATAAGTTCATGAAAAAATTGCCACGTGATGCAGAAGCTTCCAACGTGCTTGTTG
GGGAGGTTGACTTTTTGGATACTCCTTTCATTGCCTTTGTTAGGCTACAGCAGGCTGTCATG
CTGGGTGCCCTGACTGAAGTTCCTGTGCCCACAAGGTTCTTGTTCATTCTCTTAGGTCCTAA
GGGGAAAGCCAAGTCCTACCACGAGATTGGCAGAGCCATTGCCACCCTGATGTCTGATGAGG
TGTTCCATGACATTGCTTATAAAGCAAAAGACAGGCACGACCTGATTGCTGGTATTGATGAG
TTCCTAGATGAAGTCATCGTCCTTCCACCTGGGGAATGGGATCCAGCAATTAGGATAGAGCC
TCCTAAGAGTCTTCCATCCTCTGACAAAAGAAAGAATATGTACTCAGGTGGAGAGAATGTTC
AGATGAATGGGGATACGCCCCATGATGGAGGTCACGGAGGAGGAGGACATGGGGATTGTGAA
GAATTGCAGCGAACTGGACGGTTCTGTGGTGGACTAATTAAAGACATAAAGAGGAAAGCGCC
ATTTTTTGCCAGTGATTTTTATGATGCTTTAAATATTCAAGCTCTTTCGGCAATTCTCTTCA
TTTATCTGGCAACTGTAACTAATGCTATCACTTTTGGAGGACTGCTTGGGGATGCCACTGAC
AACATGCAGGGCGTGTTGGAGAGTTTCCTGGGCACTGCTGTCTCTGGAGCCATCTTTTGCCT
TTTTGCTGGTCAACCACTCACTATTCTGAGCAGCACCGGACCTGTCCTAGTTTTTGAGAGGC
TTCTATTTAATTTCAGCAAGGACAATAATTTTGACTATTTGGAGTTTCGCCTTTGGATTGGC
CTGTGGTCCGCCTTCCTATGTCTCATTTTGGTAGCCACTGATGCCAGCTTCTTGGTTCAATA
CTTCACACGTTTCACGGAGGAGGGCTTTTCCTCTCTGATTAGCTTCATCTTTATCTATGATG
CTTTCAAGAAGATGATCAAGCTTGCAGATTACTACCCCATCAACTCCAACTTCAAAGTGGGC
TACAACACTCTCTTTTCCTGTACCTGTGTGCCACCTGACCCAGCTAATATCTCAATATCTAA
TGACACCACACTGGCCCCAGAGTATTTGCCAACTATGTCTTCTACTGACATGTACCATAATA
CTACCTTTGACTGGGCATTTTTGTCGAAGAAGGAGTGTTCAAAATACGGAGGAAACCTCGTC
GGGAACAACTGTAATTTTGTTCCTGATATCACACTCATGTCTTTTATCCTCTTCTTGGGAAC
CTACACCTCTTCCATGGCTCTGAAAAAATTCAAAACTAGTCCTTATTTTCCAACCACAGCAA
GAAAACTGATCAGTGATTTTGCCATTATCTTGTCCATTCTCATCTTTTGTGTAATAGATGCC
CTAGTAGGCGTGGACACCCCAAAACTAATTGTGCCAAGTGAGTTCAAGCCAACAAGTCCAAA
CCGAGGTTGGTTCGTTCCACCGTTTGGAGAAAACCCCTGGTGGGTGTGCCTTGCTGCTGCTA
TCCCGGCTTTGTTGGTCACTATACTGATTTTCATGGACCAACAAATTACAGCTGTGATTGTA
AACAGGAAAGAACATAAACTCAAGAAAGGAGCAGGGTATCACTTGGATCTCTTTTGGGTGGC
CATCCTCATGGTTATATGCTCCCTCATGGCTCTTCCGTGGTATGTAGCTGCTACGGTCATCT
CCATTGCTCACATCGACAGTTTGAAGATGGAGACAGAGACTTCTGCACCTGGAGAACAACCA
AAGTTTCTAGGAGTGAGGGAACAAAGAGTCACTGGAACCCTTGTGTTTATTCTGACTGGTCT
GTCAGTCTTTATGGCTCCCATCTTGAAGTTTATACCCATGCCTGTACTCTATGGTGTGTTCC
TGTATATGGGAGTAGCATCCCTTAATGGTGTGCAGTTCATGGATCGTCTGAAGCTGCTTCTG
ATGCCTCTGAAGCATCAGCCTGACTTCATCTACCTGCGTCATGTTCCTCTGCGCAGAGTCCA
CCTGTTCACTTTCCTGCAGGTGTTGTGTCTGGCCCTGCTTTGGATCCTCAAGTCAACGGTGG
CTGCTATCATTTTTCCAGTAATGATCTTGGCACTTGTAGCTGTCAGAAAAGGCATGGACTAC
CTCTTCTCCCAGCATGACCTCAGCTTCCTGGATGATGTCATTCCAGAAAAGGACAAGAAAAA
GAAGGAGGATGAGAAGAAAAAGAAAAAGAAGAAGGGAAGTCTGGACAGTGACAATGATGATA
ATGGAGTCATATCCCTCATTGACTGCACTTTATTGGAGGAGCCAGAAAGCACGGAGGAGGAA
GCCAAAGGATCCGGCCAAGACATAGATCACTTGGATTTTAAAATCGGGGTGGAGCCAAAGGA
TTCCCCGCCCTTTACAGTCATCCTAGTGGCCTCGTCCAGACAGGAGAAGGCAGCGTGGACCA
GTGACATCAGCCAGTGTGTGGATAACATCCGATGCAATGGGCTCATGATGAACGCATTTGAA
GAAAATTCCAAGGTCACTGTGCCGCAGATGATCAAGTCCGACGCCTCCTTATATTGTGATGA
TGTTGACATTCGCTTCAGCAAAACCATGAACTCCTGCAAAGTGCTGCAGATCCGCTACGCCA
GTGTGGAGCGGCTGCTGGAGAGGCTGACGGACCTGCGCTTCCTGAGCATCGACTTCCTCAAC
ACCTTCCTGCACTCCTACCGCGTCTTCACCACCGCCATCGTGGTCCTGGACAAGCTCATTAC
CATCTACAAGAAGCCTATCAGTGCCATTCCTGCCAGGTGGCTGAGGTCGCTGGAGCTCCTGT
TTGCCAGTGGCCAGAACAATAAGCTCCTGTACGGTGAACCCCCCAAGTCCCCGCGCGCCACC
CGCAAGTTCTCCTCGCCGCCACCTCTGTCCATCACCAAGACATCGTCACCGAGCCGCCGGCG
GAAGCTCTCCCTGAACATCCCCATCATCACTGGCGGCAAGGCCCTGGACCTGGCCGCCCTCA
GCTGCAACTCCAATGGCTACACCAGCATGTACTCGGCCATGTCACCCTTCAGCAAGGCCACG
CTGGACACCAGCAAGCTCTATGTGTCCAGCAGCTTCACCAACAAGATTCCAGATGAGGGCGA
TACGACCCCTGAGAAGCCCGAAGACCCTTCAGCGCTCAGCAAGCAGAGCTCAGAAGTCTCCA
TGAGAGAGGAGTCAGATATTGATCAAAACCAGAGTGATGATGGTGATACTGAAACATCACCA
ACTAAATCTCCAACAACACCCAAATCAGTCAAAAACAAAAATTCTTCAGAGTTCCCACTCTT
TTCCTATAACAATGGAGTCGTCATGACCTCCTGTCGTGAACTGGACAATAACCGCAGTGCCT
TGTCGGCCGCCTCTGCCTTTGCCATAGCAACCGCCGGGGCCAACGAGGGCACCCCAAACAAG
GAGAAGTACCGGAGGATGTCCTTAGCCAGTGCAGGGTTTCCCCCAGACCAGAGGAATGGAGA
CAAGGAGTTTGTGATCCGCAGAGCAGCCACCAATCGTGTCTTGAACGTGCTCCGCCACTGGG
TGTCCAAGCACTCTCAGGACTTTGAGACCAACGATGAGCTCAAATGCAAGGTGATCGGCTTC
CTGGAAGAAGTCATGCACGACCCGGAGCTCCTGACCCAGGAGCGGAAGGCTGCAGCCAACAT
CATCAGGACTCTGACCCAGGAGGACCCAGGTGACAACCAGATCACGCTGGAGGAGATCACGC
AGATGGCTGAAGGCGTGAAGGCTGAGCCCTTTGAAAACCACTCAGCCCTGGAGATCGCGGAG
CAGCTGACCCTGCTAGATCACCTCGTCTTCAAGAAGATTCCTTATGAGGAGTTCTTCGGACA
AGGATGGATGAAACTGGAAAAGAATGAAAGGACCCCTTATATCATGAAAACCACTAAGCACT
TCAATGACATCAGTAACTTGATTGCTTCAGAAATCATCCGCAATGAGGACATCAACGCCAGG
GTGAGCGCCATCGAGAAGTGGGTGGCCGTAGCTGACATATGCCGCTGCCTCCACAACTACAA
TGCCGTACTGGAGATCACCTCGTCCATGAACCGCAGTGCAATCTTCCGGCTCAAAAAGACGT
GGCTCAAAGTCTCTAAGCAGACTAAAGCTTTGATTGATAAGCTCCAAAAGCTTGTGTCATCT
GAGGGCAGATTTAAGAATCTCAGAGAAGCTCTGAAAAATTGTGACCCACCCTGTGTCCCTTA
CCTGGGGATGTACCTCACCGACCTGGCCTTCATCGAGGAGGGGACGCCCAATTACACGGAAG
ACGGCCTGGTCAACTTCTCCAAGATGAGGATGATATCCCATATTATCCGAGAGATTCGCCAG
TTTCAACAAACTGCCTACAAAATAGAGCACCAAGCAAAGGTAACGCAATATTTACTGGACCA
ATCTTTTGTAATGGATGAAGAAAGCCTCTACGAGTCTTCTCTCCGAATAGAACCAAAACTCC
CCACCTGA
IQGAP1-RASGRF1 cDNA (RASGRF1 sequence is)
(SEQ ID NO: 13)
ATGTCCGCCGCAGACGAGGTTGACGGGCTGGGCGTGGCCCGGCCGCACTATGGCTCTGTCCT
GGATAATGAAAGACTTACTGCAGAGGAGATGGATGAAAGGAGACGTCAGAACGTGGCTTATG
AGTACCTTTGTCATTTGGAAGAAGCGAAGAGGTCCGACGCCTCCTTATATTGTGATGATGTT
GACATTCGCTTCAGCAAAACCATGAACTCCTGCAAAGTGCTGCAGATCCGCTACGCCAGTGT
GGAGCGGCTGCTGGAGAGGCTGACGGACCTGCGCTTCCTGAGCATCGACTTCCTCAACACCT
TCCTGCACTCCTACCGCGTCTTCACCACCGCCATCGTGGTCCTGGACAAGCTCATTACCATC
TACAAGAAGCCTATCAGTGCCATTCCTGCCAGGTCGCTGGAGCTCCTGTTTGCCAGTGGCCA
GAACAATAAGCTCCTGTACGGTGAACCCCCCAAGTCCCCGCGCGCCACCCGCAAGTTCTCCT
CGCCGCCACCTCTGTCCATCACCAAGACATCGTCACCGAGCCGCCGGCGGAAGCTCTCCCTG
AACATCCCCATCATCACTGGCGGCAAGGCCCTGGACCTGGCCGCCCTCAGCTGCAACTCCAA
TGGCTACACCAGCATGTACTCGGCCATGTCACCCTTCAGCAAGGCCACGCTGGACACCAGCA
AGCTCTATGTGTCCAGCAGCTTCACCAACAAGATTCCAGATGAGGGCGATACGACCCCTGAG
AAGCCCGAAGACCCTTCAGCGCTCAGCAAGCAGAGCTCAGAAGTCTCCATGAGAGAGGAGTC
AGATATTGATCAAAACCAGAGTGATGATGGTGATACTGAAACATCACCAACTAAATCTCCAA
CAACACCCAAATCAGTCAAAAACAAAAATTCTTCAGAGTTCCCACTCTTTTCCTATAACAAT
GGAGTCGTCATGACCTCCTGTCGTGAACTGGACAATAACCGCAGTGCCTTGTCGGCCGCCTC
TGCCTTTGCCATAGCAACCGCCGGGGCCAACGAGGGCACCCCAAACAAGGAGAAGTACCGGA
GGATGTCCTTAGCCAGTGCAGGGTTTCCCCCAGACCAGAGGAATGGAGACAAGGAGTTTGTG
ATCCGCAGAGCAGCCACCAATCGTGTCTTGAACGTGCTCCGCCACTGGGTGTCCAAGCACTC
TCAGGACTTTGAGACCAACGATGAGCTCAAATGCAAGGTGATCGGCTTCCTGGAAGAAGTCA
TGCACGACCCGGAGCTCCTGACCCAGGAGCGGAAGGCTGCAGCCAACATCATCAGGACTCTG
ACCCAGGAGGACCCAGGTGACAACCAGATCACGCTGGAGGAGATCACGCAGATGGCTGAAGG
CGTGAAGGCTGAGCCCTTTGAAAACCACTCAGCCCTGGAGATCGCGGAGCAGCTGACCCTGC
TAGATCACCTCGTCTTCAAGAAGATTCCTTATGAGGAGTTCTTCGGACAAGGATGGATGAAA
CTGGAAAAGAATGAAAGGACCCCTTATATCATGAAAACCACTAAGCACTTCAATGACATCAG
TAACTTGATTGCTTCAGAAATCATCCGCAATGAGGACATCAACGCCAGGGTGAGCGCCATCG
AGAAGTGGGTGGCCGTAGCTGACATATGCCGCTGCCTCCACAACTACAATGCCGTACTGGAG
ATCACCTCGTCCATGAACCGCAGTGCAATCTTCCGGCTCAAAAAGACGTGGCTCAAAGTCTC
TAAGCAGACTAAAGCTTTGATTGATAAGCTCCAAAAGCTTGTGTCATCTGAGGGCAGATTTA
AGAATCTCAGAGAAGCTCTGAAAAATTGTGACCCACCCTGTGTCCCTTACCTGGGGATGTAC
CTCACCGACCTGGCCTTCATCGAGGAGGGGACGCCCAATTACACGGAAGACGGCCTGGTCAA
CTTCTCCAAGATGAGGATGATATCCCATATTATCCGAGAGATTCGCCAGTTTCAACAAACTG
CCTACAAAATAGAGCACCAAGCAAAGGTAACGCAATATTTACTGGACCAATCTTTTGTAATG
GATGAAGAAAGCCTCTACGAGTCTTCTCTCCGAATAGAACCAAAACTCCCCACCTGA

### Example 3: RASGRF1 Fusions Increase GTP-RAS, Activate RAS Signaling, and Promote Cell Transformation

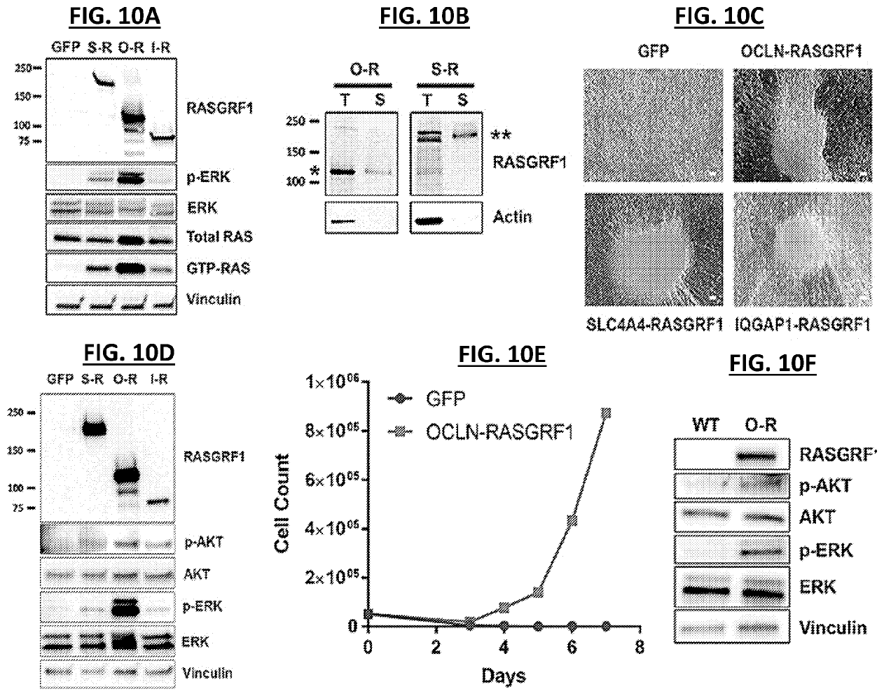

The full-length cDNAs were cloned into a lentiviral expression vector and expressed in HEK 293T cells. An antibody against the C-terminus of RASGRF1 was used to confirm ectopic expression of the fusion proteins (FIG. 10A). Given the function of the C-terminal RAS-GEF domain of RASGRF1, it was hypothesized that expression of RASGRF1 fusions increases levels of GTP-bound (active) RAS. Using an affinity purification assay for GTP-RAS, we observed a marked increase in levels of GTP-RAS in HEK 293T cells expressing OCLN-RASGRF1, SLC4A4-RASGRF1, or IQGAP1-RASGRF1 compared to green fluorescent protein (GFP; FIG. 10A). Furthermore, RAF-MEK-ERK signaling was upregulated with expression of RASGRF1 fusions (FIG. 10A). These effects appeared more robust with OCLN-RAGSRF1 and SLC4A4-RASGRF1 compared to IQGAP1-RASGRF1.

As the N-terminal protein in both OCLN-RASGRF1 and SLC4A4-RASGRF1 is a transmembrane protein, it was then investigated whether these two fusions can be detected at the cell surface. Using surface protein biotinylation and purification, labeled cell surface RASGRF1 fusions were isolated from lysates of HEK 293T cells overexpressing OCLN-RASGRF1 and from PaCaDD137 cells with endogenous SLC4A4-RASGRF1 (FIG. 10B).

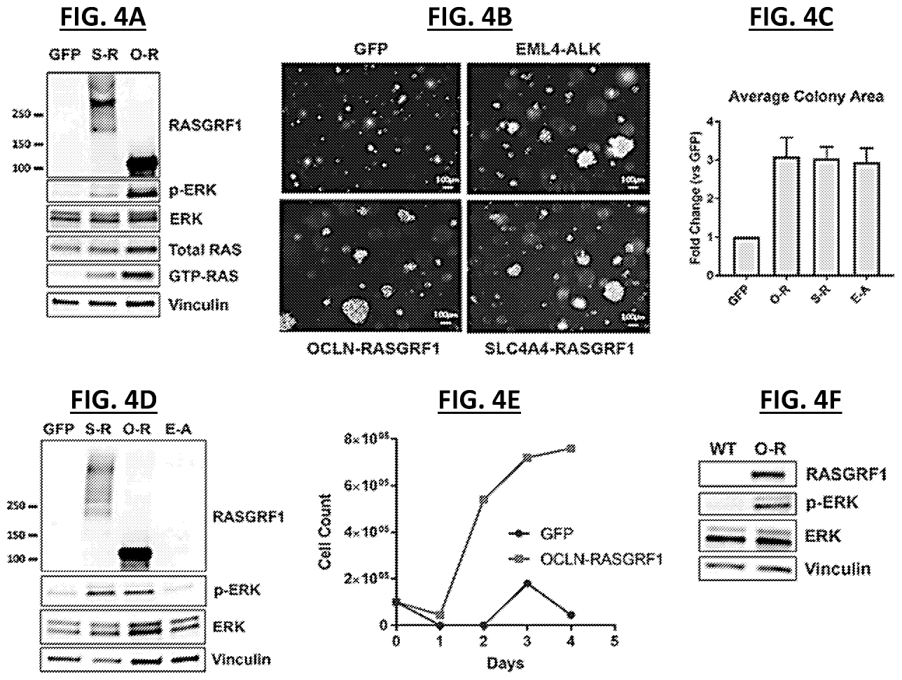

It was next determined if RASGRF1 fusions promote cellular transformation. RASGRF1 fusions were expressed in NIH3T3 mouse fibroblasts and assayed anchorage-independent proliferation in soft agar. NIH3T3 cells expressing GFP or the established oncogenic fusion EML4-ALK were used as negative and positive controls, respectively. Like EML4-ALK, we observed that ectopic expression of OCLN-RASGRF1 and SLC4A4-RASGRF1 induced robust colony formation in soft agar with a 3-fold increase in average colony size compared to GFP (FIGS. 4B-4C). IQGAP1-RASGRF1 did not promote anchorage-independent proliferation in these assays. However, all 3 RASGRF1 fusions did promote proliferation with loss of contact inhibition leading to tumor cell foci formation in NIH3T3 cells (FIG. 10C). These findings suggest OCLN-RASGRF1 and SLC4A4-RASGRF1 may have enhanced transforming potential compared to IQGAP1-RASGRF1. Ectopic expression of all 3 RASGRF1 fusions activated the RAF-MEK-ERK and PI3K pathways, downstream effectors of RAS signaling (FIG. 10D).

OCLN-RASGRF1 was then expressed in the interleukin-3 (IL-3)-dependent Ba/F3 hematopoietic cell line to determine if OCLN-RASGRF1 induces IL-3-independent proliferation. We maintained Ba/F3 cells expressing OCLN-RASGRF1 or GFP in 1 ng/mL IL-3. Over the course of approximately 3 weeks, IL-3 concentrations were weaned to 0.001 ng/mL before withdrawing IL-3 from growth media entirely. Ba/F3 cells expressing OCLN-RASGRF1 (but not GFP) demonstrated robust proliferation in the absence of IL-3 (FIG. 10E). Ectopic expression of OCLN-RASGRF1 in Ba/F3 cells was confirmed with Western immunoblotting and was associated with RAF-MEK-ERK and PI3K activation (FIG. 10F).

### Example 4: Cancer Cells Expressing RASGRF1 Fusions are Sensitive to RAF-MEK-ERK Inhibition

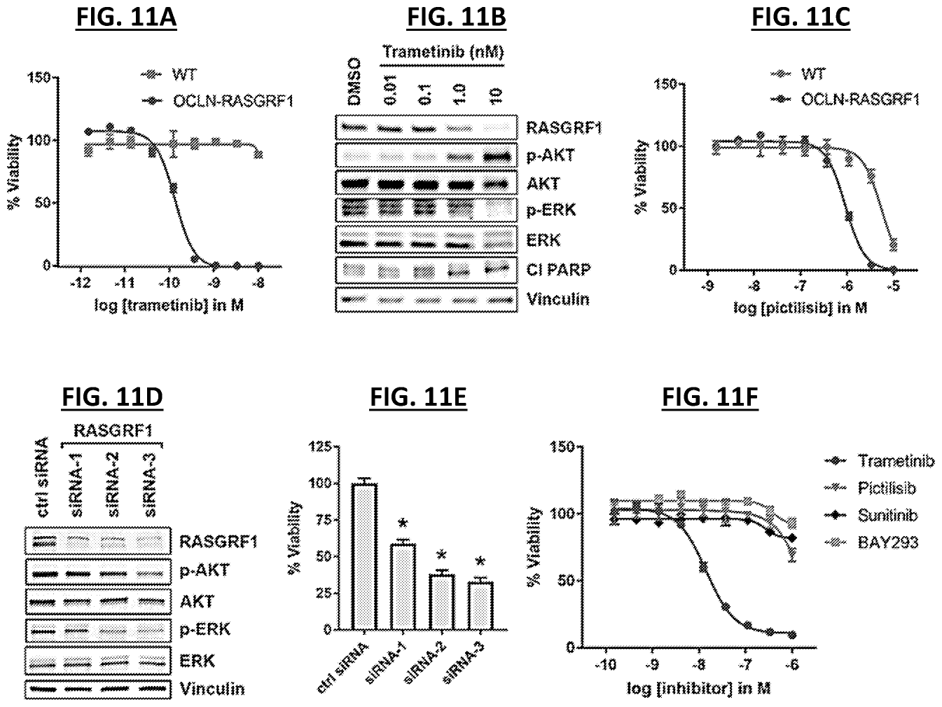

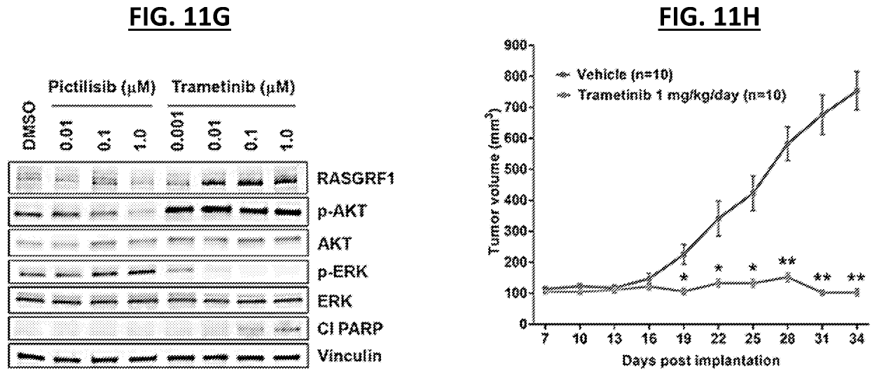

The sensitivity of Ba/F3 cells driven by OCLN-RASGRF1 to inhibitors of the RAF-MEK-ERK and PI3K pathways was then evaluated. It was observed that Ba/F3 cells expressing OCLN-RASGRF1 (but not wild-type Ba/F3 cells) were highly sensitive to the MEK inhibitor trametinib with a half maximal inhibitory concentration (IC50) of approximately 150 pM (FIG. 11A). Treatment with trametinib induced apoptosis as evidenced by induction of PARP cleavage (FIG. 11B). In contrast, Ba/F3 cells expressing OCLN-RASGRF1 showed only a modest increase in sensitivity to the pan-PI3K inhibitor pictilisib relative to wild-type cells (FIG. 1 IC).

Studies then used siRNAs targeting RASGRF1 to reduce SLC4A4-RASGRF1 levels in PaCaDD137 cells. Depletion of SLC4A4-RASGRF1 was associated with a reduction in RAF-MEK-ERK and (to a lesser extent) PI3K signaling together with impaired cell viability (FIG. 1D-11E). These findings implicate SLC4A4-RASGRF1 as a dependency and potential therapeutic target in PaCaDD137 cells.

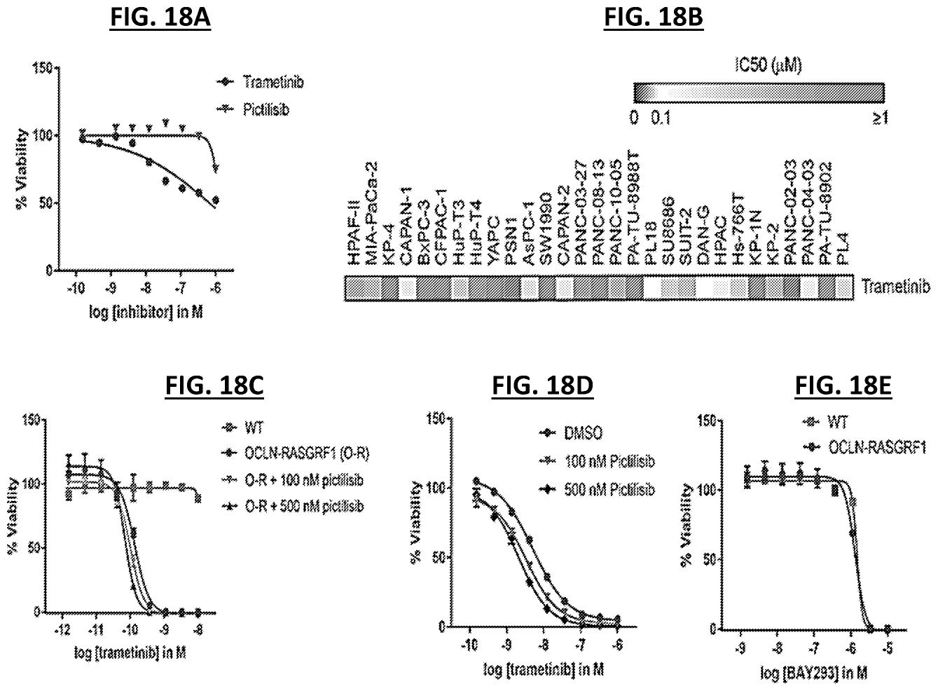

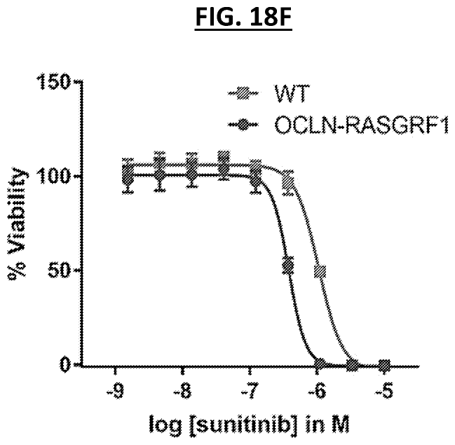

It was observed that PaCaDD137 cells are highly sensitive to trametinib (IC50 ˜20 nM) but not pictilisib (FIG. 1 IF). Like Ba/F3 cells with ectopic OCLN-RASGRF1, treatment of PaCaDD137 cells with trametinib induced apoptosis (FIG. 1 IG). Studies then examined the sensitivity of other PDAC cell lines to trametinib. In contrast to PaCaDD137, the KRAS-mutant PDAC cell line SU8686 has limited sensitivity to trametinib (FIG. 18A). To more broadly assess the sensitivity of PDAC cell lines to trametinib, we queried data from the Genomics of Drug Sensitivity in Cancer Project (GDSC2 dataset; cancerrxgene.org). Of 29 PDAC cell lines profiled with trametinib, only 3 (all KRAS-mutant) demonstrated an IC50<100 nM (FIG. 18B). Without wishing to be bound by theory, these findings suggest PaCaDD137 cells are generally more sensitive to MEK inhibition than most PDAC cell lines. The addition of pictilisib to trametinib showed only a modest increase in activity compared to trametinib alone in Ba/F3 cells expressing OCLN-RASGRF1 and in PaCaDD137 cells (FIG. 18C-18D).

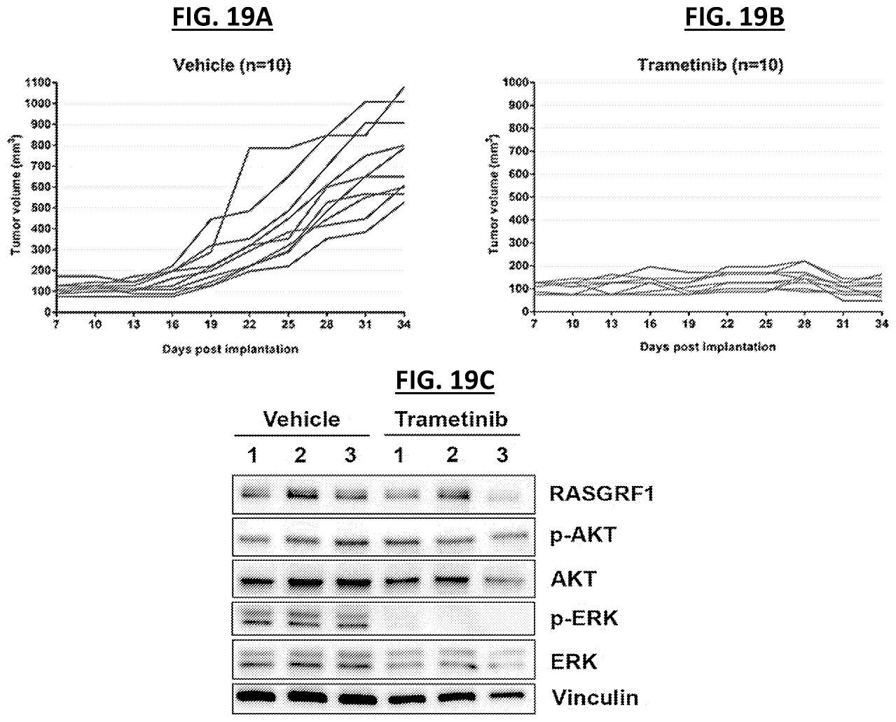

Given the potent activity of trametinib in PaCaDD137 cells harboring SLC4A4-RASGRF1, it was then tested whether trametinib impairs proliferation of PaCaDD137 cells in a xenograft model. PaCaDD137 xenografts were established by subcutaneous injection into the flanks of nude mice. Treatment with trametinib (1 mg/kg/day) resulted in marked tumor growth inhibition compared to vehicle (FIG. 11H, FIG. 19). Without wishing to be bound by theory, these findings suggest RAF-MEK-ERK inhibition as a potential therapeutic strategy for tumors harboring RASGRF1 fusions.

### Example 5: Selected Discussion

It is reported herein that over 90% of lung adenocarcinomas from never-smokers or individuals with <10 pack-year smoking history in a single-institution cohort harbor an established oncogenic driver. This represents a higher proportion than prior published reports in North American and European populations, likely due to the growing number of driver oncogenes identified in recent years. In addition to frequent driver oncogenes in LUADs from never-smokers, nearly all (39 of 40) LUADs from patients with a light smoking history in our study harbored an established oncogenic driver. Notably, most driver alterations identified in the present study are actionable with FDA-approved targeted therapies currently available for alterations identified in 73 of the 103 patients (71%). In addition, promising molecular therapies for NSCLC harboring KRAS G12C are currently being evaluated in clinical studies. These findings underscore the critical importance of comprehensive molecular profiling of lung adenocarcinomas (especially among never-smokers and individuals with minimal smoking history) as these studies can have important treatment implications in the advanced disease setting.

A novel in-frame OCLN-RASGRF1 gene fusion associated with increased RASGRF1 expression compared to control tumors was identified and cloned from 1 of the 5 tumors in the cohort that lacked an established oncogenic driver. A similar fusion, SLC4A4-RASGRF1, was identified from the pancreatic cancer cell line PaCaDD137. It was demonstrated herein that these fusions increase endogenous levels of activated GTP-bound RAS, activate MAP kinase signaling, and promote cell transformation. As approximately 90% of pancreatic adenocarcinomas harbor activating KRAS alterations, it is notable that KRAS is not mutated in PaCaDD137 (Rückert, F. et al., J. Surg. Res., 2012, 172:29-39; Buscail, L. et al., Nat. Rev. Gastroenterol. Hepatol., 2020, 17:153-168). This raises the possibility that PaCaDD137 could represent an RAS-driven tumor mediated by RASGRF1 upregulation rather than a primary activating alteration in KRAS itself. Further studies will be necessary to explore this possibility. Indeed, other oncogenic gene fusions including NRG1 and ROS1 fusions have been identified in pancreatic adenocarcinomas without KRAS mutations (Jones, M. R. et al., Clin. Cancer Res., 2019, 25:4674-4681; Aguirre, A. J. et al., Cancer Discov., 2018, 8:1096-1111). These findings motivate the search for other RASGRF1 fusions in pancreatic adenocarcinomas (particularly those that lack activating KRAS mutations).

In addition to the three RASGRF1 fusions presented herein, case reports of similar fusions involving RASGRF1 have been described. A TMEM154-RASGRF1 fusion containing Exons 15-28 of RASGRF1 has been reported in a patient with a relapsed acute myeloid malignancy (Watts, J. M. et al., Int. J. Mol. Sci., 2017:1492). More recently, a TMEM87A-RASGRF1 fusion containing Exons 8-28 of RASGRF1 was reported from a never-smoker with advanced lung adenocarcinoma (Cooper, A. J. et al., Clin. Cancer Res., 2020). Of note, this latter patient reportedly experienced an exceptional therapeutic response to the multi-kinase inhibitor sunitinib although the molecular basis of this response was not defined. No sensitivity of Ba/F3 cells expressing OCLN-RASGRF1 or of PaCaDD137 cells to sunitinib was observed herein (FIG. 5B).

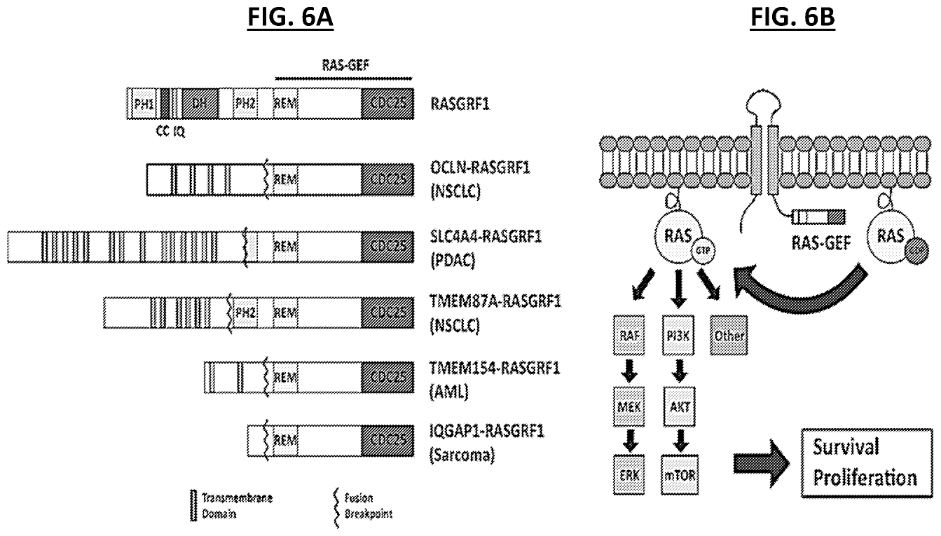

Strikingly, all 4 of these RASGRF1 fusions feature a transmembrane protein as the 5′ fusion partner with the rearrangement occurring in a location predicted to anchor the RAS-GEF domain of RASGRF1 to the cell membrane within a carboxy-terminal cytoplasmic tail (FIG. 6). As membrane association is required for RAS activation, the 5′ transmembrane fusion partner may serve to localize the RAS-GEF catalytic domain to membrane-associated RAS and thus enhance activation (FIG. 6B). Consistent with this, prior studies have shown that targeting of the RASGRF1 catalytic domain to the cell membrane is sufficient to promote RAS activation and transformation. Herein it is demonstrated that OCLN-RASGRF1 and SLC4A4-RASGRF1 can be detected at the cell surface. Of note, these findings suggest IQGAP1-RASGRF1 (which is not predicted to span the plasma membrane) displays a weaker transforming phenotype than OCLN-RASGRF1 and SLC4A4-RASGRF1. Further studies will be required for more comprehensive characterization of the localization of RASGRF1 fusion proteins. While it is unclear which specific RAS isoforms are activated by RASGRF1 fusions, prior studies suggest wild-type RASGRF1 preferentially activates HRAS compared to KRAS or NRAS.

The present findings indicate that gene fusions involving the RAS-GEF domain of RASGRF1 represent a recurrent driver alteration in cancer. Of note, activating somatic mutations in the GEF SOS1 have been described in NSCLC, suggesting multiple potential mechanisms of GEF-mediated oncogenic RAS activation in cancer. Additional studies will be required to establish the frequency of RASGRF1 fusions in NSCLC, PDAC, and other malignancies. The findings presented herein nominate the RAF-MEK-ERK pathway as a potential therapeutic target in RASGRF1-rearranged tumors. This work may also provide a rationale to capture RASGRF1 fusions with comprehensive tumor molecular profiling platforms as potentially actionable alterations in cancer. More generally, these findings highlight the potential of genomic characterization of LUADs from light and never-smokers without established oncogenic drivers to uncover previously unrecognized oncogenic alterations that may be relevant for multiple malignancies.

### ENUMERATED EMBODIMENTS

The following enumerated embodiments are provided, the numbering of which should not be construed as designating levels of importance.

Embodiment 1 provides a method of treating a subject having cancer, the method comprising administering a treatment for cancer to a pre-selected subject, wherein the subject is pre-selected by determining that an RASGRF1 gene fusion is present in a sample obtained from the subject.

Embodiment 2 provides a method of diagnosing cancer in a subject, the method comprising determining the presence or absence of an RASGRF1 gene fusion in a sample obtained from the subject, wherein the presence of an RASGRF1 gene fusion indicates that the subject has cancer.

Embodiment 3 provides a method of identifying a subject having cancer who is responsive to a therapy directed against RASGRF1 or a pathway activated by RASGRF1, the method comprising determining the presence or absence of an RASGRF1 gene fusion in a sample obtained from the subject, wherein the presence of an RASGRF1 gene fusion indicates the subject is responsive to a therapy directed against RASGRF1 or a pathway activated by RASGRF1.

Embodiment 4 provides the method of any one of embodiments 1-3, wherein the sample is a tissue sample, blood sample, or a tumor sample.

Embodiment 5 provides the method of any one of embodiments 1-4, wherein the RASGRF1 gene fusion comprises at least a portion of the RASGRF1 gene fused to at least a portion of a transmembrane protein.

Embodiment 6 provides the method of embodiment 5, wherein the transmembrane protein is selected from the group consisting of: OCLN, TMEM87A, SLC4A4, and TMEM154.

Embodiment 7 provides the method of any one of embodiments 1-6, wherein the RASGRF1 gene fusion comprises the nucleotide sequence set forth in SEQ ID NOs: 11, 12, or 13.

Embodiment 8 provides the method of any one of embodiments 1-7, wherein the cancer is non-small cell lung carcinoma, pancreatic cancer, or bone marrow cancer.

Embodiment 9 provides the method of any one of embodiments 2-8, wherein the step of determining the presence or absence of the RASGRF1 gene fusion in the sample comprises detecting the presence or absence of RASGRF1 gene fusion in the sample by whole-exome sequencing, whole-transcriptome sequencing, RNA sequencing, fluorescence in situ hybridization, immunohistochemistry, and/or a combination thereof.

Embodiment 10 provides the method of embodiment 2 or 3, further comprising a step of selecting and/or administering a treatment to the subject identified as having cancer or the subject identified as responsive to a therapy directed against RASGRF1 or a pathway activated by RASGRF1.

Embodiment 11 provides the method of any one of embodiments 1, 3, or 10, wherein the treatment or therapy comprises a MAP kinase inhibitor, a MEK inhibitor, a PI3K inhibitor, a tyrosine kinase inhibitor (TKI), an inhibitor of the Ras-GEF domain of RASGRF1, an inhibitor of the GEF family, an ERK inhibitor, or combinations thereof.

Embodiment 12 provides the method of embodiment 11, wherein the treatment or therapy is selected from the group consisting of: imatinib, gefitinib, erlotinib, dasatinib, sunitinib, adavosertib, lapatinib, efametinib, selumetinib, trametinib, cobimetinib, idelalisib, copanlisib, duvelisib, alpelisib, taselisib, perifosine, buparlisib, umbralisib, voxtalisib, pictilisib, BAY-293, BI 1701963, BI-3406, ulixertinib, LY3214996, CC-90003, AZD0364, S0859, and combinations thereof.

Embodiment 13 provides the method of any one of embodiments 1, 3, or 10, wherein the treatment disrupts or prevents the RASGRF1 gene fusion.

Embodiment 14 provides a kit for the diagnosis cancer or for the identification of a subject responsive to a therapy directed against RASGRF1 or a pathway activated by RASGRF1, the kit comprising at least one agent capable of specifically binding or hybridizing to a polypeptide or polynucleotide of an RASGRF1 fusion, and directions for using the agent for the diagnosis of cancer.

Embodiment 15 provides the kit of embodiment 14, further comprising directions and/or materials necessary for detecting the presence of fusion between at least a portion of an RASGRF1 polypeptide or polynucleotide and at least a portion of a second polypeptide or polynucleotide.

Embodiment 16 provides the kit of embodiment 15, wherein the fusion is between at least a portion of the RASGRF1 polypeptide or polynucleotide and at least a portion of an OCLN, a TMEM87A, a SLC4A4, or a TMEM154 polypeptide or polynucleotide.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of treating a pre-selected subject having cancer,
the method comprising administering a treatment for cancer to a pre-selected subject,
wherein the subject is pre-selected by determining that an RASGRF1 gene fusion is present in a sample obtained from the subject.

2. A method of diagnosing cancer in a subject,
the method comprising determining the presence or absence of an RASGRF1 gene fusion in a sample obtained from the subject,
wherein the presence of an RASGRF1 gene fusion indicates that the subject has cancer.

3. The method of claim 2
wherein the presence of the RASGRF1 gene fusion indicates the subject is responsive to a therapy directed against RASGRF1 or a pathway activated by RASGRF1.

4. The method of claim 1, wherein the sample is a tissue sample, blood sample, or a tumor sample.

5. The method of claim 1, wherein the RASGRF1 gene fusion comprises at least a portion of the RASGRF1 gene fused to at least a portion of a transmembrane protein.

6. The method of claim 5, wherein the transmembrane protein is selected from the group consisting of OCLN, TMEM87A, SLC4A4, and TMEM154.

7. The method of claim 1, wherein the RASGRF1 gene fusion comprises the nucleotide sequence set forth in SEQ ID NO: 11, 12, or 13.

8. The method of claim 1, wherein the cancer is non-small cell lung carcinoma, pancreatic cancer, and/or bone marrow cancer.

9. The method of claim 2, wherein the step of determining the presence or absence of the RASGRF1 gene fusion in the sample comprises detecting the presence or absence of RASGRF1 gene fusion in the sample by at least one of whole-exome sequencing, whole-transcriptome sequencing, RNA sequencing, fluorescence in situ hybridization, and immunohistochemistry.

10. The method of claim 2, further comprising a step of selecting and/or administering a treatment to the subject identified as having cancer or the subject identified as responsive to a therapy directed against RASGRF1 or a pathway activated by RASGRF1.

11. The method of claim 1, wherein the treatment or therapy comprises at least one of a MAP kinase inhibitor, a MEK inhibitor, a PI3K inhibitor, a tyrosine kinase inhibitor (TKI), an inhibitor of the Ras-GEF domain of RASGRF1, an inhibitor of the GEF family, and an ERK inhibitor.

12. The method of claim 11, wherein the treatment or therapy is selected from the group consisting of: imatinib, gefitinib, erlotinib, dasatinib, sunitinib, adavosertib, lapatinib, efametinib, selumetinib, trametinib, cobimetinib, idelalisib, copanlisib, duvelisib, alpelisib, taselisib, perifosine, buparlisib, umbralisib, voxtalisib, pictilisib, BAY-293, BI 1701963, BI-3406, ulixertinib, LY3214996, CC-90003, AZD0364, S0859, and combinations thereof.

13. The method of claim 1, wherein the treatment disrupts or prevents the RASGRF1 gene fusion.

14. A kit for the diagnosis of cancer or for the identification of a subject responsive to a therapy directed against RASGRF1 or a pathway activated by RASGRF1, the kit comprising at least one agent capable of specifically binding or hybridizing to a polypeptide or polynucleotide of an RASGRF1 fusion, and directions for using the agent for the diagnosis of cancer.

15. The kit of claim 14, further comprising directions or materials necessary for detecting the presence of fusion between at least a portion of an RASGRF1 polypeptide or polynucleotide and at least a portion of a second polypeptide or polynucleotide.

16. The kit of claim 15, wherein the fusion is between at least a portion of the RASGRF1 polypeptide or polynucleotide and at least a portion of an OCLN, a TMEM87A, a SLC4A4, or a TMEM154 polypeptide or polynucleotide.


SEQUENCE LISTING (20 sequences)

SEQ ID NO: 1 - DNA, 25 bp, synthetic construct
  source 1..25: mol_type = other DNA
catcctgaagatcagctgaccattg

SEQ ID NO: 2 - DNA, 32 bp, synthetic construct
  source 1..32: mol_type = other DNA
cgaatatgtacagtatcatctagcacatgtcc

SEQ ID NO: 3 - DNA, 64 bp, synthetic construct
  source 1..64: mol_type = other DNA
ggggacaactttgtacaaaaaagttggcaccccgccatgtcatccaggcctcttgaaagtccac

SEQ ID NO: 4 - DNA, 57 bp, synthetic construct
  source 1..57: mol_type = other DNA
ggggacaactttgtacaagaaagttggcaatcaggtggggagttttggttctattcg

SEQ ID NO: 5 - DNA, 57 bp, synthetic construct
  source 1..57: mol_type = other DNA
ggggacaactttgtacaaaaaagttggcaccatggaggatgaagctgtcctggacag

SEQ ID NO: 6 - DNA, 58 bp, synthetic construct
  source 1..58: mol_type = other DNA
ggggacaactttgtacaaaaaagttggcaccatgtccgccgcagacgaggttgacggg

SEQ ID NO: 7 - DNA, 30 bp, synthetic construct
  source 1..30: mol_type = other DNA
ctataaatccacgccgtccgacgcctcctt

SEQ ID NO: 8 - DNA, 30 bp, synthetic construct
  source 1..30: mol_type = other DNA
agtgacaatgatgataatggagtcatatcc

SEQ ID NO: 9 - DNA, 24 bp, synthetic construct
  source 1..24: mol_type = other DNA
aaatccacgccgtccgacgcctcc

SEQ ID NO: 10 - DNA, 24 bp, synthetic construct
  source 1..24: mol_type = other DNA
gacaatgatgataatggagtcata

SEQ ID NO: 11 - DNA, 2985 bp, synthetic construct
  source 1..2985: mol_type = other DNA
atgtcatccaggcctcttgaaagtccacctccttacaggcctgatgaattcaaaccgaatcattatgcaccaagcaatgacatatatggtggagagatgcatgttcgaccaatgctctctcagccagcctactctttttacccagaagatgaaattcttcacttctacaaatggacctctcctccaggagtgattcggatcctgtctatgctcattattgtgatgtgcattgccatctttgcctgtgtggcctccacgcttgcctgggacagaggctatggaacttcccttttaggaggtagtgtaggctacccttatggaggaagtggctttggtagctacggaagtggctatggctatggctatggttatggctatggctacggaggctatacagacccaagagcagcaaagggcttcatgttggccatggctgccttttgtttcattgccgcgttggtgatctttgttaccagtgttataagatctgaaatgtccagaacaagaagatactacttaagtgtgataatagtgagtgctatcctgggcatcatggtgtttattgccacaattgtctatataatgggagtgaacccaactgctcagtcttctggatctctatatggttcacaaatatatgccctctgcaaccaattttatacacctgcagctactggactctacgtggatcagtatttgtatcactactgtgttgtggatccccaggaggccattgccattgtactggggttcatgattattgtggcttttgctttaataattttctttgctgtgaaaactcgaagaaagatggacaggtatgacaagtccaatattttgtgggacaaggaacacatttatgatgagcagccccccaatgtcgaggagtgggttaaaaatgtgtctgcaggcacacaggacgtgccttcacccccatctgactatgtggaaagagttgacagtcccatggcatactcttccaatggcaaagtgaatgacaagcggttttatccagagtcttcctataaatccacgccgtccgacgcctccttatattgtgatgatgttgacattcgcttcagcaaaaccatgaactcctgcaaagtgctgcagatccgctacgccagtgtggagcggctgctggagaggctgacggacctgcgcttcctgagcatcgacttcctcaacaccttcctgcactcctaccgcgtcttcaccaccgccatcgtggtcctggacaagctcattaccatctacaagaagcctatcagtgccattcctgccaggtcgctggagctcctgtttgccagtggccagaacaataagctcctgtacggtgaaccccccaagtccccgcgcgccacccgcaagttctcctcgccgccacctctgtccatcaccaagacatcgtcaccgagccgccggcggaagctctccctgaacatccccatcatcactggcggcaaggccctggacctggccgccctcagctgcaactccaatggctacaccagcatgtactcggccatgtcacccttcagcaaggccacgctggacaccagcaagctctatgtgtccagcagcttcaccaacaagattccagatgagggcgatacgacccctgagaagcccgaagacccttcagcgctcagcaagcagagctcagaagtctccatgagagaggagtcagatattgatcaaaaccagagtgatgatggtgatactgaaacatcaccaactaaatctccaacaacacccaaatcagtcaaaaacaaaaattcttcagagttcccactcttttcctataacaatggagtcgtcatgacctcctgtcgtgaactggacaataaccgcagtgccttgtcggccgcctctgcctttgccatagcaaccgccggggccaacgagggcaccccaaacaaggagaagtaccggaggatgtccttagccagtgcagggtttcccccagaccagaggaatggagacaaggagtttgtgatccgcagagcagccaccaatcgtgtcttgaacgtgctccgccactgggtgtccaagcactctcaggactttgagaccaacgatgagctcaaatgcaaggtgatcggcttcctggaagaagtcatgcacgacccggagctcctgacccaggagcggaaggctgcagccaacatcatcaggactctgacccaggaggacccaggtgacaaccagatcacgctggaggagatcacgcagatggctgaaggcgtgaaggctgagccctttgaaaaccactcagccctggagatcgcggagcagctgaccctgctagatcacctcgtcttcaagaagattccttatgaggagttcttcggacaaggatggatgaaactggaaaagaatgaaaggaccccttatatcatgaaaaccactaagcacttcaatgacatcagtaacttgattgcttcagaaatcatccgcaatgaggacatcaacgccagggtgagcgccatcgagaagtgggtggccgtagctgacatatgccgctgcctccacaactacaatgccgtactggagatcacctcgtccatgaaccgcagtgcaatcttccggctcaaaaagacgtggctcaaagtctctaagcagactaaagctttgattgataagctccaaaagcttgtgtcatctgagggcagatttaagaatctcagagaagctctgaaaaattgtgacccaccctgtgtcccttacctggggatgtacctcaccgacctggccttcatcgaggaggggacgcccaattacacggaagacggcctggtcaacttctccaagatgaggatgatatcccatattatccgagagattcgccagtttcaacaaactgcctacaaaatagagcaccaagcaaaggtaacgcaatatttactggaccaatcttttgtaatggatgaagaaagcctctacgagtcttctctccgaatagaaccaaaactccccacctga

SEQ ID NO: 12 - DNA, 5340 bp, synthetic construct
  source 1..5340: mol_type = other DNA
atggaggatgaagctgtcctggacagaggggcttccttcctcaagcatgtgtgtgatgaagaagaagtagaaggccaccataccatttacatcggagtccatgtgccgaagagttacaggagaaggagacgtcacaagagaaagacagggcacaaagaaaagaaggaaaaggagagaatctctgagaactactctgacaaatcagatattgaaaatgctgatgaatccagcagcagcatcctaaaacctctcatctctcctgctgcagaacgcatccgattcatcttgggagaggaggatgacagcccagctccccctcagctcttcacggaactggatgagctgctggccgtggatgggcaggagatggagtggaaggaaacagccaggtggatcaagtttgaagaaaaagtggaacagggtggggaaagatggagcaagccccatgtggccacattgtcccttcatagtttatttgagctgaggacatgtatggagaaaggatccatcatgcttgatcgggaggcttcttctctcccacagttggtggagatgattgttgaccatcagattgagacaggcctattgaaacctgaacttaaggataaggtgacctatactttgctccggaagcaccggcatcaaaccaagaaatccaaccttcggtccctggctgacattgggaagacagtctccagtgcaagtaggatgtttaccaaccctgataatggtagcccagccatgacccataggaatctgacttcctccagtctgaatgacatttctgataaaccggagaaggaccagctgaagaataagttcatgaaaaaattgccacgtgatgcagaagcttccaacgtgcttgttggggaggttgactttttggatactcctttcattgcctttgttaggctacagcaggctgtcatgctgggtgccctgactgaagttcctgtgcccacaaggttcttgttcattctcttaggtcctaaggggaaagccaagtcctaccacgagattggcagagccattgccaccctgatgtctgatgaggtgttccatgacattgcttataaagcaaaagacaggcacgacctgattgctggtattgatgagttcctagatgaagtcatcgtccttccacctggggaatgggatccagcaattaggatagagcctcctaagagtcttccatcctctgacaaaagaaagaatatgtactcaggtggagagaatgttcagatgaatggggatacgccccatgatggaggtcacggaggaggaggacatggggattgtgaagaattgcagcgaactggacggttctgtggtggactaattaaagacataaagaggaaagcgccattttttgccagtgatttttatgatgctttaaatattcaagctctttcggcaattctcttcatttatctggcaactgtaactaatgctatcacttttggaggactgcttggggatgccactgacaacatgcagggcgtgttggagagtttcctgggcactgctgtctctggagccatcttttgcctttttgctggtcaaccactcactattctgagcagcaccggacctgtcctagtttttgagaggcttctatttaatttcagcaaggacaataattttgactatttggagtttcgcctttggattggcctgtggtccgccttcctatgtctcattttggtagccactgatgccagcttcttggttcaatacttcacacgtttcacggaggagggcttttcctctctgattagcttcatctttatctatgatgctttcaagaagatgatcaagcttgcagattactaccccatcaactccaacttcaaagtgggctacaacactctcttttcctgtacctgtgtgccacctgacccagctaatatctcaatatctaatgacaccacactggccccagagtatttgccaactatgtcttctactgacatgtaccataatactacctttgactgggcatttttgtcgaagaaggagtgttcaaaatacggaggaaacctcgtcgggaacaactgtaattttgttcctgatatcacactcatgtcttttatcctcttcttgggaacctacacctcttccatggctctgaaaaaattcaaaactagtccttattttccaaccacagcaagaaaactgatcagtgattttgccattatcttgtccattctcatcttttgtgtaatagatgccctagtaggcgtggacaccccaaaactaattgtgccaagtgagttcaagccaacaagtccaaaccgaggttggttcgttccaccgtttggagaaaacccctggtgggtgtgccttgctgctgctatcccggctttgttggtcactatactgattttcatggaccaacaaattacagctgtgattgtaaacaggaaagaacataaactcaagaaaggagcagggtatcacttggatctcttttgggtggccatcctcatggttatatgctccctcatggctcttccgtggtatgtagctgctacggtcatctccattgctcacatcgacagtttgaagatggagacagagacttctgcacctggagaacaaccaaagtttctaggagtgagggaacaaagagtcactggaacccttgtgtttattctgactggtctgtcagtctttatggctcccatcttgaagtttatacccatgcctgtactctatggtgtgttcctgtatatgggagtagcatcccttaatggtgtgcagttcatggatcgtctgaagctgcttctgatgcctctgaagcatcagcctgacttcatctacctgcgtcatgttcctctgcgcagagtccacctgttcactttcctgcaggtgttgtgtctggccctgctttggatcctcaagtcaacggtggctgctatcatttttccagtaatgatcttggcacttgtagctgtcagaaaaggcatggactacctcttctcccagcatgacctcagcttcctggatgatgtcattccagaaaaggacaagaaaaagaaggaggatgagaagaaaaagaaaaagaagaagggaagtctggacagtgacaatgatgataatggagtcatatccctcattgactgcactttattggaggagccagaaagcacggaggaggaagccaaaggatccggccaagacatagatcacttggattttaaaatcggggtggagccaaaggattccccgccctttacagtcatcctagtggcctcgtccagacaggagaaggcagcgtggaccagtgacatcagccagtgtgtggataacatccgatgcaatgggctcatgatgaacgcatttgaagaaaattccaaggtcactgtgccgcagatgatcaagtccgacgcctccttatattgtgatgatgttgacattcgcttcagcaaaaccatgaactcctgcaaagtgctgcagatccgctacgccagtgtggagcggctgctggagaggctgacggacctgcgcttcctgagcatcgacttcctcaacaccttcctgcactcctaccgcgtcttcaccaccgccatcgtggtcctggacaagctcattaccatctacaagaagcctatcagtgccattcctgccaggtggctgaggtcgctggagctcctgtttgccagtggccagaacaataagctcctgtacggtgaaccccccaagtccccgcgcgccacccgcaagttctcctcgccgccacctctgtccatcaccaagacatcgtcaccgagccgccggcggaagctctccctgaacatccccatcatcactggcggcaaggccctggacctggccgccctcagctgcaactccaatggctacaccagcatgtactcggccatgtcacccttcagcaaggccacgctggacaccagcaagctctatgtgtccagcagcttcaccaacaagattccagatgagggcgatacgacccctgagaagcccgaagacccttcagcgctcagcaagcagagctcagaagtctccatgagagaggagtcagatattgatcaaaaccagagtgatgatggtgatactgaaacatcaccaactaaatctccaacaacacccaaatcagtcaaaaacaaaaattcttcagagttcccactcttttcctataacaatggagtcgtcatgacctcctgtcgtgaactggacaataaccgcagtgccttgtcggccgcctctgcctttgccatagcaaccgccggggccaacgagggcaccccaaacaaggagaagtaccggaggatgtccttagccagtgcagggtttcccccagaccagaggaatggagacaaggagtttgtgatccgcagagcagccaccaatcgtgtcttgaacgtgctccgccactgggtgtccaagcactctcaggactttgagaccaacgatgagctcaaatgcaaggtgatcggcttcctggaagaagtcatgcacgacccggagctcctgacccaggagcggaaggctgcagccaacatcatcaggactctgacccaggaggacccaggtgacaaccagatcacgctggaggagatcacgcagatggctgaaggcgtgaaggctgagccctttgaaaaccactcagccctggagatcgcggagcagctgaccctgctagatcacctcgtcttcaagaagattccttatgaggagttcttcggacaaggatggatgaaactggaaaagaatgaaaggaccccttatatcatgaaaaccactaagcacttcaatgacatcagtaacttgattgcttcagaaatcatccgcaatgaggacatcaacgccagggtgagcgccatcgagaagtgggtggccgtagctgacatatgccgctgcctccacaactacaatgccgtactggagatcacctcgtccatgaaccgcagtgcaatcttccggctcaaaaagacgtggctcaaagtctctaagcagactaaagctttgattgataagctccaaaagcttgtgtcatctgagggcagatttaagaatctcagagaagctctgaaaaattgtgacccaccctgtgtcccttacctggggatgtacctcaccgacctggccttcatcgaggaggggacgcccaattacacggaagacggcctggtcaacttctccaagatgaggatgatatcccatattatccgagagattcgccagtttcaacaaactgcctacaaaatagagcaccaagcaaaggtaacgcaatatttactggaccaatcttttgtaatggatgaagaaagcctctacgagtcttctctccgaatagaaccaaaactccccacctga

SEQ ID NO: 13 - DNA, 2103 bp, synthetic construct
  source 1..2103: mol_type = other DNA
atgtccgccgcagacgaggttgacgggctgggcgtggcccggccgcactatggctctgtcctggataatgaaagacttactgcagaggagatggatgaaaggagacgtcagaacgtggcttatgagtacctttgtcatttggaagaagcgaagaggtccgacgcctccttatattgtgatgatgttgacattcgcttcagcaaaaccatgaactcctgcaaagtgctgcagatccgctacgccagtgtggagcggctgctggagaggctgacggacctgcgcttcctgagcatcgacttcctcaacaccttcctgcactcctaccgcgtcttcaccaccgccatcgtggtcctggacaagctcattaccatctacaagaagcctatcagtgccattcctgccaggtcgctggagctcctgtttgccagtggccagaacaataagctcctgtacggtgaaccccccaagtccccgcgcgccacccgcaagttctcctcgccgccacctctgtccatcaccaagacatcgtcaccgagccgccggcggaagctctccctgaacatccccatcatcactggcggcaaggccctggacctggccgccctcagctgcaactccaatggctacaccagcatgtactcggccatgtcacccttcagcaaggccacgctggacaccagcaagctctatgtgtccagcagcttcaccaacaagattccagatgagggcgatacgacccctgagaagcccgaagacccttcagcgctcagcaagcagagctcagaagtctccatgagagaggagtcagatattgatcaaaaccagagtgatgatggtgatactgaaacatcaccaactaaatctccaacaacacccaaatcagtcaaaaacaaaaattcttcagagttcccactcttttcctataacaatggagtcgtcatgacctcctgtcgtgaactggacaataaccgcagtgccttgtcggccgcctctgcctttgccatagcaaccgccggggccaacgagggcaccccaaacaaggagaagtaccggaggatgtccttagccagtgcagggtttcccccagaccagaggaatggagacaaggagtttgtgatccgcagagcagccaccaatcgtgtcttgaacgtgctccgccactgggtgtccaagcactctcaggactttgagaccaacgatgagctcaaatgcaaggtgatcggcttcctggaagaagtcatgcacgacccggagctcctgacccaggagcggaaggctgcagccaacatcatcaggactctgacccaggaggacccaggtgacaaccagatcacgctggaggagatcacgcagatggctgaaggcgtgaaggctgagccctttgaaaaccactcagccctggagatcgcggagcagctgaccctgctagatcacctcgtcttcaagaagattccttatgaggagttcttcggacaaggatggatgaaactggaaaagaatgaaaggaccccttatatcatgaaaaccactaagcacttcaatgacatcagtaacttgattgcttcagaaatcatccgcaatgaggacatcaacgccagggtgagcgccatcgagaagtgggtggccgtagctgacatatgccgctgcctccacaactacaatgccgtactggagatcacctcgtccatgaaccgcagtgcaatcttccggctcaaaaagacgtggctcaaagtctctaagcagactaaagctttgattgataagctccaaaagcttgtgtcatctgagggcagatttaagaatctcagagaagctctgaaaaattgtgacccaccctgtgtcccttacctggggatgtacctcaccgacctggccttcatcgaggaggggacgcccaattacacggaagacggcctggtcaacttctccaagatgaggatgatatcccatattatccgagagattcgccagtttcaacaaactgcctacaaaatagagcaccaagcaaaggtaacgcaatatttactggaccaatcttttgtaatggatgaagaaagcctctacgagtcttctctccgaatagaaccaaaactccccacctga

SEQ ID NO: 14 - DNA, 178 bp, synthetic construct
  source 1..178: mol_type = other DNA
atgtccgccgcagacgaggttgacgggctgggcgtggcccggccgcactatggctctgtcctggataatgaaagacttactgcagaggagatggatgaaaggagacgtcagaacgtggcttatgagtacctttgtcatttggaagaagcgaagaggtccgacgcctccttatattgtg

SEQ ID NO: 15 - protein, 8 aa, synthetic construct
EAKRSDAS

SEQ ID NO: 16 - DNA, 24 bp, synthetic construct
  source 1..24: mol_type = other DNA
gaagcgaagaggtccgacgcctcc

SEQ ID NO: 17 - protein, 8 aa, synthetic construct
KSTPSDAS

SEQ ID NO: 18 - protein, 8 aa, synthetic construct
DNDDNGVI

SEQ ID NO: 19 - protein, 10 aa, synthetic construct
YKSTPSDASL

SEQ ID NO: 20 - protein, 10 aa, synthetic construct
SDNDDNGVIS